(19) United States
(12) Patent Application Publication
(10) Pub. No.: US 2009/0155337 A1
(43) Pub. Date: Jun. 18, 2009

(54) METHOD AND AGENT FOR IN-SITU STABILIZATION OF VASCULAR TISSUE

(75) Inventors: Stefan G. Schreck, Fallbrook, CA (US); Kemal Schankereli, Stillwater, MN (US)

Correspondence Address: KNOBBE MARTENS OLSON & BEAR LLP, 2040 MAIN STREET, FOURTEENTH FLOOR, IRVINE, CA, 92614, US

(73) Assignee: ENDOLOGIX, INC., Irvine, CA (US)

(21) Appl. No.: 12/269,677

(22) Filed: Nov. 12, 2008

Related U.S. Application Data
(60) Provisional application No. 60/987,268, filed on Nov. 12, 2007.
(60) Provisional application No. 60/987,261, filed on Nov. 12, 2007.
(60) Provisional application No. 61/012,356, filed on Dec. 7, 2007.
(60) Provisional application No. 61/127,654, filed on May 14, 2008.
(60) Provisional application No. 61/012,579, filed on Dec. 10, 2007.

(51) Int. Cl. A61F 2/00; A61K 31/352; A61K 33/06

(52) U.S. Cl. 424/426; 514/456; 424/693

(57) ABSTRACT

A method for stabilizing an extra cellular matrix layer in the vascular system of the body is disclosed herein. The method can comprise placing a vascular catheter adjacent to the extra cellular matrix layer, delivering a solution containing a bioflavonoid to the extra cellular matrix layer with the vascular catheter, and cross-linking protein in the extra cellular matrix layer. The bioflavonoid can be a catechin, particularly epigallocatechin gallate (EGCG).

### PRIORITY INFORMATION

This application also claims priority benefit under 35 U.S.C. § 119(e) of Provisional Application 60/987,268 filed Nov. 12, 2007, Provisional Application 60/987,261 filed Nov. 12, 2007, Provisional Application 61/012,356 filed Dec. 7, 2007, Provisional Application 61/127,654 filed May 14, 2008, and Provisional Application 61/012,579 filed Dec. 10, 2007, which applications are hereby incorporated by reference as if fully set forth herein.

### BACKGROUND OF THE INVENTION

1. Field of the Disclosure

The present disclosure relates to therapeutic agents and delivery methods for in-situ stabilization of vascular tissue

2. Background of the Disclosure

Cardiovascular disease is one of the leading causes of death in the developed countries. It is estimated that more than one million people in the United States suffer from a sudden cardiac event each year. For a long time, coronary artery occlusions have been believed to be the main cause of sudden cardiac events. The occlusion of coronary arteries reduces the blood flow to the myocardium. In cases of severe occlusion and high cardiac workload, myocardial muscle cells do not receive sufficient oxygen and die. Clinical interventions for occlusion of coronary artery have focused on removing the blockage in the arteries. This is accomplished by expanding the artery with a balloon (balloon angioplasty), placement of stents in the lesion to keep the artery patent, or coronary bypass surgery with a vein graft. Despite the effectiveness of these procedures in treating stenotic lesions, patients still suffer from sudden cardiac events even in the absence of stenotic lesions.

Over the past several years, the attention of research into sudden cardiac events has shifted to vulnerable plaque, a rupture-prone plaque in the walls of coronary arteries. Vulnerable plaque is characterized by a large lipid pool in the plaque, a thin fibrous cap separating the plaque from the blood stream, and an inflammatory process within the plaque. Macrophages that infiltrate the fibrous cap can break down the collagen structure of the cap by enzymatic degradation. The cap can become too weak to withstand high hemodynamic loads and can ultimately rupture, exposing the highly thrombogenic content of the plaque to the bloodstream. Thrombi can form rapidly and can cause partial or complete occlusion of the blood vessel. It is believed that vulnerable plaque may be responsible for as many as 60-80% of all sudden cardiac events.

Vulnerable plaque is not only found in the coronary artery but in the whole arterial system of “vulnerable patients.” Rupture of vulnerable plaque in the ascending aorta is believed to be a main cause of stroke as the thrombus released from the plaque travels through the carotid arteries into the brain. Vulnerable plaque has also been found in the carotid arteries themselves. Approximately 600,000 Americans suffer a stroke each year. One-third die within one year and another one-third have severe disability.

Vulnerable plaque is difficult to research because current imaging systems are not capable of detecting the plaque in the vessel wall. Therefore, the investigation into vulnerable plaque has been limited to the biophysical and biochemical analysis of cadavers and retrospective studies of patients who have suffered a sudden cardiac event. Only some of the key findings of the ongoing research into vulnerable plaque are highlighted here.

Lipid-rich plaques seem to exist throughout the coronary and vascular systems of high-risk patients with “hot spots” of increased inflammatory activities. The size of vulnerable plaque in coronary arteries is typically less than 1 cm in length and covers approximately one-quarter to one-half of the circumference of the blood vessel. The fibrous cap can have a thickness of less than 100 μm. A slight stenosis of the vessel may be present in some cases. Rupture of plaque seems to increase during periods of elevated physical activity or mental stress. Retrospective studies have identified several patient specific risk factors associated with sudden cardiac death. They include hypercoagulable blood, presence of serum markers of atherosclerosis and inflammation, and pre-existing atherosclerosis-related myocardial damage.

Advances in the detection of vulnerable plaque has spurned a need and opportunity for local or regional treatment modalities. Drug-eluding stents have been proposed for treatment of vulnerable plaque. Although vessel occlusion is not critical in vulnerable plaque, stents can support the thin fibrous cap while applying time-released drugs to suppress the inflammatory reaction. One major shortcoming of stenting is the need for several stents in cases of multiple or diffuse lesions and the high cost of drug-eluding stents.

Aortic aneurysm disease involves the tissue of the aortic vessel. Over-expression of enzymes (matrix metalloproteinase) can break down the elastin and collagen structure in the wall. The vessel wall can become weak and expand radially and axially in response to blood pressure. Degradation of the collagen structure can ultimately lead to aortic rupture and potential patient death.

Additionally, one of the primary failure modes of small-diameter surgical grafts are thrombosis of the lumen and stenosis due to hyperplasia at the anastomosis site. It is believed that thrombosis is due to the limited hemocompatibility of the graft material in high-shear flows. Hyperplasia is typically triggered by the mechanical injury and stresses introduced into the tissue at the surgical anastomosis. Attempts have been made to improve the hemocompatibility of the graft material by providing a porous surface to promote ingrowth, biocompatible coatings (e.g. carbon (BIOLITE), collagen (HEMASHIELD)) or bioactive coatings (e.g. heparin (GORE PROPATEN)). To address the issue of hyperplasia, specific graft designs (e.g. Bard VENAFLO) or drug-treatment with anti-hyperplastic agent (e.g. Paclitaxel (Angiotech VASCULAR WRAP)) have been proposed.

U.S. Pat. Nos. 5,295,962 and 5,569,184 describe a drug delivery and dilation catheter for the delivery of a therapeutic agent before, during, or after balloon dilatation. The design of the catheter is well suited for the continuous infusion of a therapeutic agent, but lacks control of delivery of a single dose of a therapeutic agent into the vessel wall.

There is an obvious need for the controlled delivery of a single dose of therapeutic agent into the vessel wall. Particularly, for the delivery of agents of known toxicity and/or where the therapeutic effect of the agent is highly dose dependent. The current disclosure provides solutions to this issue.

### SUMMARY OF THE DISCLOSURE

Catechins can be applied to local vessel injuries to promote healing, restore normal function of the endothelium, reduce thrombosis, stabilize the extra-cellular matrix via cross-linking and inhibition of enzymatic degradation, reduce inflammation, and inhibit smooth muscle cell proliferation. Injuries to the vessel wall can be caused by atherosclerosis, vulnerable plaque, angioplasty, stent placement, atherectomy, surgical anestomosis, and endovascular devices. It will be obvious to one of ordinary skill in the art that catechin can be used for treating a wide range of local vessel injuries or diseases.

Accordingly, some embodiments of the present disclosure are directed to a method for stabilizing an extra cellular matrix layer in the vascular system of the body comprising: placing a vascular catheter adjacent to the extra cellular matrix layer; delivering a solution containing a bioflavonoid to the extra cellular matrix layer with the vascular catheter; and cross-linking protein in the extra cellular matrix layer. In some arrangements, the protein can be a collagen and the bioflavonoid can be a proanthocyanidin, catechin, epicatechin, epigallo catechin, epicatechin gallate, epigallocatechin gallate, quercetin, tannic acid, or any combination thereof. The extra cellular matrix layer can be located in an aortic aneurysm, or in any location where such a condition may occur.

Some embodiments of the present disclosure are directed to a solution for treating an extra cellular matrix layer in situ, comprising a bioflavonoid and a keotropic agent. The bioflavonoid can be selected from the group consisting of: proanthocyanidin, catechin, epicatechin, epigallo catechin, epicatechin gallate, epigallocatechin gallate, quercetin, tannic acid, and any combination thereof, and the keotropic agent can be Ca(OH)₂. Further, the solution can be used to treat an extra cellular matrix layer located in an injured or diseased artery, for example, but not limited to, a stenotic artery.

Some embodiments are directed to a method for reducing hyperplesia after injury of a blood vessel, comprising: placing a vascular catheter adjacent to a wall of the blood vessel; and delivering a solution containing a bioflavonoid to the wall of the blood vessel with the vascular catheter. The bioflavonoid can be, but is not required to be, selected from the group consisting of: proanthocyanidin, catechin, epicatechin, epigallo catechin, epicatechin gallate, epigallocatechin gallate, quercetin, tannic acid, and any combination thereof.

Additionally, some embodiments comprise a method of releasing a therapeutic agent such as, but not limited to, catechins, to a location within the body of an animal. In some embodiments, the therapeutic agent can be coated onto the surface of delivery device; the coated surface of the delivery device can be placed in contact with the treatment site; a second agent can be administered to the surface through the delivery device; and the second agent releases the therapeutic agent from the surface of the delivery device.

Other embodiments comprise a device for the release of a therapeutic agent into the body of an animal, wherein a therapeutic agent can be coated onto the surface of the device. The device includes means of placing the coated surface in contact with the treatment site, and the device includes a lumen to deliver a release agent to the coated surface.

Other embodiments comprise a device for the release of a therapeutic agent into the body. The device includes a catheter body comprising at least one moveable wall that carries at least partially with a therapeutic agent; the moveable wall being either porous or permeable. The catheter defines a fluid path in fluid communication with the moveable wall to permit a release agent to reach the therapeutic agent carried by the moveable wall.

Other embodiments comprise a method of releasing a therapeutic agent to a location within the body of an animal. The method includes advancing a catheter with a membrane that carries therapeutic agent; positioning the membrane against a tissue surface; and injecting a expressing release agent through the catheter and the membrane to release therapeutic agent into the tissue.

Others embodiments disclosed herein pertain to the rapid release of a therapeutic agent into animal tissue. In some embodiments, the surface layer of the drug delivery system that can be used to release a therapeutic agent into animal tissue can comprise a polymer or elastomer that contains the therapeutic agent. To release the drug, the layer can be stretched to increase the permeability of the layer and generate micro channels for the drug to exit the layer. Specifically, some embodiments comprise an angioplasty balloon catheter. The balloon can be immersed in a solvent containing a therapeutic agent to absorb the agent. When the balloon is inflated against the arterial wall, the balloon material can be stretched and the agent can be released and transferred into the aortic wall.

Some embodiments of the present disclosure are directed to a method of releasing a therapeutic agent to a treatment site within the body of an animal, comprising coating the therapeutic agent is coated onto the surface of a delivery device; placing the coated surface of the delivery device in contact with the treatment site; administering a second agent to the surface through the delivery device so as to release the therapeutic agent from the surface of the delivery device to the treatment site.

Some embodiments are directed to a device configured to release a therapeutic agent into the body, the device comprising a catheter body comprising at least one moveable wall that at least partially carries a therapeutic agent; the moveable wall being either porous or permeable; and a fluid path in fluid communication with the moveable wall to permit a release agent to reach the therapeutic agent carried by the moveable wall.

Other embodiments comprise improving the biocompatibility of a medical device, specifically ePTFE vascular graft, by loading the graft material with an anti-thrombogenic and/or anti-hyperplastic agent. ePTFE grafts consist of a matrix of PTFE fibers and nodes. The density of the material and degree of porosity can be controlled during the manufacturing process. In some embodiments, the open space in the ePTFE structure can be used to store a therapeutic agent that modifies the surface kinetics of the ePTFE to prevent or reduce thrombosis. In another embodiment, the open space in the ePTFE structure can be used to store a therapeutic agent that can be released into the adjacent tissue after implantation to reduce smooth muscle cell proliferation and hyperplasia of the blood vessel, or aneurysm growth.

Additionally, in some embodiments, the agent can also be applied in conjunction with traditional endovascular stent grafts in the treatment of aortic aneurysms. For example, the distal and proximal seal area of the stent graft can be stabilized by applying the agent to the adjacent tissue to prevent dilation of the aorta in the seal zone.

Some embodiments are directed to an apparatus for delivering a therapeutic agent to an extra cellular matrix layer in the vascular system of the body, the apparatus comprising an expandable member configured to be expanded against the extra cellular matrix layer and a bioflavonoid carried by the expandable member, wherein the expandable member is configured to release the bioflavonoid into the extra cellular matrix layer when the expandable member is expanded against the extra cellular matrix layer. In some arrangements, the expandable member can be a stent graft that can, but is not required to, comprise ePTFE. In some arrangements, the expandable member can comprise at least one expandable balloon configured to be expanded against the extra cellular matrix layer, the balloon optionally comprising latex. In some arrangements, the expandable member can comprise an expandable balloon and a drug carrying member, the drug carrying member at least partially covering the expandable balloon and configured to carry the bioflavonoid and selectively release the bioflavonoid into the extra cellular matrix layer when the drug carrying member is positioned adjacent to the extra cellular matrix layer and expanded by the expandable balloon.

### BRIEF DESCRIPTION OF THE DRAWINGS

These and other features, aspects and advantages of the present disclosure will now be described in connection with non-exclusive embodiments, in reference to the accompanying drawings. The illustrated embodiments, however, are merely examples and are not intended to limit the invention. The following are brief descriptions of the drawings, which may not be drawn to scale.

FIG. 1 illustrates the molecular structure of various catechins.

FIG. 2 is a partial sectional side view of one arrangement of a drug delivery and temporary stent catheter.

FIG. 3 is a cross-sectional view taken along the lines 3-3 of FIG. 2.

FIG. 4 is a partial sectional side view of another arrangement of a catheter, having a coaxially configured catheter body.

FIG. 5 is a cross-sectional view taken along the lines 5-5 in FIG. 4.

FIG. 6 is a partial sectional side view of an over-the-wire arrangement of a catheter.

FIG. 7 is a partial sectional side view of a non-stent arrangement of a catheter.

FIG. 8 is a cross-sectional view taken along the lines 8-8 in FIG. 7.

FIG. 9 is a cross-sectional view taken along the lines 9-9 in FIG. 7.

FIG. 10 is a cross-sectional view taken along the lines 10-10 in FIG. 7.

FIG. 11 is a side view of a non-stent arrangement in communication with a fluid delivery and guide-wire entry apparatus.

FIG. 12 is a perspective view of the non-stent embodiment the catheter.

FIG. 13 is a schematic illustration of an embodiment of an angioplasty balloon catheter that can comprise semi-elastic balloon loaded with a therapeutic agent.

FIG. 14A is a schematic illustration of an embodiment of an angioplasty balloon catheter comprising a PTA balloon covered by a tubular sleeve that can be loaded with a therapeutic agent, showing the balloon in a collapsed state.

FIG. 14B is a schematic illustration of the embodiment of the angioplasty balloon catheter shown in FIG. 14A, showing the PTA balloon in a partially inflated state.

FIG. 14C is a schematic illustration of the embodiment of the angioplasty balloon catheter shown in FIG. 14A, showing the balloon in a fully inflated state.

FIG. 15A is a schematic illustration of another embodiment of an angioplasty balloon catheter comprising an inner PTA balloon and an outer balloon that can be loaded with a therapeutic agent, showing the balloons in a collapsed state.

FIG. 15B is a schematic illustration of the embodiment of the angioplasty balloon catheter shown in FIG. 15A, showing the balloons in a fully inflated state.

FIG. 16A is a schematic illustration of an angioplasty balloon catheter with an inner PTA balloon and an outer balloon that can be loaded with a therapeutic agent, showing the inner balloon inflating the distal section of the outer balloon.

FIG. 16B is a schematic illustration of the embodiment of the angioplasty balloon catheter shown in FIG. 16A, showing the inner balloon inflating the proximal section of the outer balloon.

FIG. 17A shows an SEM image at 5.0 k magnification of a latex surface prepared with 1% PEG having molecular weight of between approximately 380-420.

FIG. 17B shows an SEM image at 5.0 k magnification of the surface of the latex shown in FIG. 17A, stretched to about 400% of its original dimensions

### DETAILED DESCRIPTION OF THE PREFERRED EMBODIMENTS

The following detailed description is now directed to certain specific embodiments of the disclosure. In this description, reference is made to the drawings wherein like parts are designated with like numerals throughout the description and the drawings.

As mentioned above, vulnerable plaque is characterized by a large lipid pool in the plaque, a thin fibrous cap separating the plaque from the blood stream, and an inflammatory process within the plaque. Disclosed herein are, inter alia, apparatuses, methods, and other treatment details for local treatment of vulnerable plaque that targets the thin fibrous cap. Research indicates that the fibrous cap can be eroded by enzymatic degradation of the protein in the cap that ultimately causes the plaque to rupture. It is postulated that rupture of the plaque could be prohibited or at least significantly delayed by cross-linking the collagen in the fibrous cap to prevent enzymatic degradation.

The chemical agent used to cross-link the collagen in the fibrous cap and inhibit expression of degrading enzymes can be selected from tannic acid or the family of bio-flavonoids, preferably from the group of catechins. These flavonoids are also referred to as proanthocyanidins or condensed tannins.

Catechins contain OH functions in their molecular structure that can form hydrogen bonds with protein in the body. Although hydrogen bonds are less stable than covalent bonds, the ability of catechin to create multiple bonds with complex proteins substantially increases the strength of the cross-links. It can be therefore desirable to choose catechins with a large number of OH functions in its structure. For example, adding a gallate molecule to the base structure of catechin adds two additional OH functions to the overall molecular structure. Epicatechin gallate (ECG) and epigallocatechin gallate (EGCG) show a high affinity to complex protein structures such as collagen.

Arterial aneurysms are typically caused by the enzymatic degradation of elastin and collagen in the vessel wall resulting in a weakening, dilatation, and potential rupture of the artery. Cross-linking of the collagen and elastin by catechin can prevent the development and rupture of the aneurysm. There are other chronic inflammatory diseases that are associated with enzymatic degradation of protein such as Alzheimer disease and arthritis. Thus, the stabilization of protein by cross-linking with catechin may have application in many other diseases.

Catechin can also reduce hyperplasia after aortic wall injury caused by angioplasty balloons. Two mechanisms are believed to be involved. Catechins seem to effect the proliferation of cells directly by chemically bonding to components within the cells. In addition, catechins cross-link the collagen matrix making it more resistant to cell infiltration and migration. In another embodiment, catechins can also create a negative charge similar to magnesium sulphate. An additional beneficial effect of cross-linking the vessel wall with catechins may be reduced thrombogenicity.

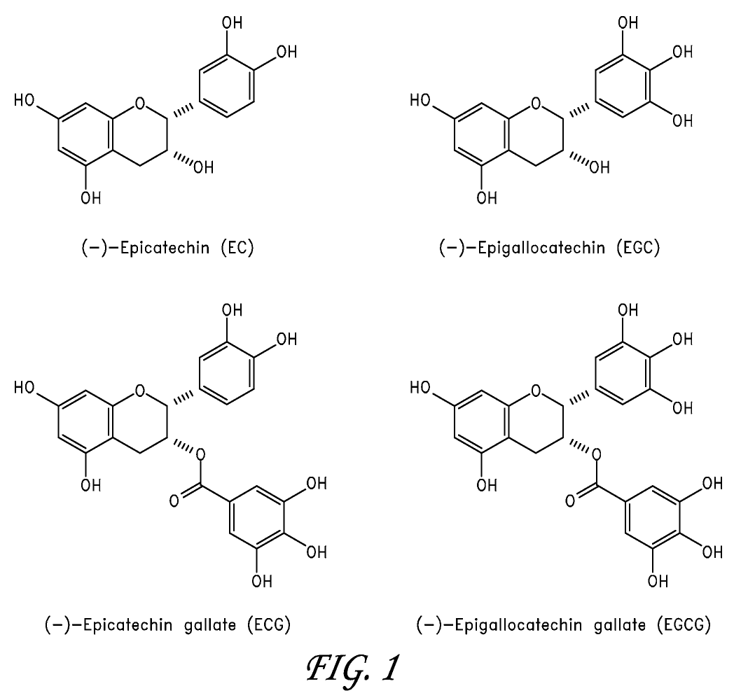

FIG. 1 illustrates the molecular structure of several catechins that can be useful to cross-link the collagen in the fibrous cap. Alternatively, catechins can, for example, be combined with copper to form a catechin-copper complex. The catechin-copper can form aldehyde groups that then react with amino acids in elastin or collagen such as lysine and arginine to generate stable cross-links. The fibrous cap typically has a high content of collagen allowing for stabilization of the cap by delivering a solution containing catechin via a drug delivery catheter directly to the vessel wall.

The present disclosure involves methods of treating vulnerable plaque by cross-linking the fibrous cap, or collagenous extra cellular matrix layer, on the inner wall or vascular intima of the vessel. The techniques described herein may produce some change in the underlying lipid pool, but the primary intent is protecting or strengthening the fibrous cap to help prevent future rupture thereof. The methods of the present disclosure are believed to be superior to those that treat the lipid pool because an area of the extra cellular matrix layer larger than the underlying lipid pool can be treated with the methods of the present disclosure. This can help protect against ruptures of later forming vulnerable plaque deposits closely adjacent to the first deposits.

Stabilization of tissue in-situ requires that the reaction produces biocompatible, non-cytotoxic reaction products. A group of biocompatible bioflavenoids have been identified that contain OH functions, which form hydrogen bonds with proteins. The preferred embodiment of the agent contains predominantly epigallocatechin gallate (EGCG) and Epicatechin Gallate (ECG), which generally have a high affinity for collagen.

The bioflavonoid EGCG or other suitable agents to can be delivered in a pH-buffered solution (e.g. phosphate buffer) of pH 7.4 to minimize damage to living tissue. The concentration of the agents can be in the range from approximately 0.01% to approximately 5% preferably between 0.1% and 1.0%. In some cases it may be advantageous to alter the pH of the solution in order to optimize the reaction kinetics. For example, hydrogen bonds form about 50% faster when the pH is reduced to 4.0. The reaction kinetics may be affected by the pH of the treatment solution compared to the isoelectric point of the protein to be cross-linked. It may be advantageous to match the pH of the solution to the isoelectric point of collagen (pH 5.6-5.8) or elastin (pH 4.0) in the vessel. To improve penetration of the agent into the tissue and minimize swelling of the tissue during fixation, a keotropic agent such as Ca(OH)₂ or Dimethyl Salfoxite (DMSO) can be added to the solution.

P B Dobrin et al. describes in “Elastolytic and Collagenolytic Studies of Arteries. Implications for the Mechanical Properties of Aneurysms” (Arch Surg. 1984, 119(4): 405-9) in vitro studies performed on arteries from humans and animals. As described therein, the author treated human and animal arteries with collagenase and elastase. From the experiments, the author concluded that loss of elastin caused canine arteries to dilate but the arteries remained intact. Loss of elastin in human arteries only caused a slight dilatation of the arteries. The arteries also stayed intact. However, loss of collagen caused both human and animal arteries to rupture.

S Menashi et al. describes in “Collagen in Abdominal Aortic Aneurysm: Typing, Content, and Degradation” (Journal of Vascular Surgery, 1987, 6(6)) measurements of the collagen content in human abdominal aortas. The author found that the collagen concentration increases in aortic aneurysms. The author also notes a high collagenase activity level in ruptured aneurysms.

In “Increased Turnover of Collagen in Abdominal Aortic Aneurysms, Demonstrated by Measuring the Concentration of Aminoterminal Propeptide of Type III Procollagen in Peripheral and Aortal Blood Samples” (Journal of Vascular Surgery 1995, 22(2), J Satta et al. provide further clarification on the observations by Menashi, referenced above. J Satta et al. measured an increased turnover of collagen in aortic aneurysms. According to the authors, the turnover is due to simultaneous degradation of collagen by enzymes and increased synthesis of collagen in response to increasing wall stresses.

In “Collagen Types and Matrix Protein Content in Human Abdominal Aortic Aneurysm” (Journal of Vascular Surgery, 1989, 10(4)), R J Rizzo et al. measured the collagen and elastin content in aortic aneurysm and compared the results to healthy control groups. The authors measured elastin concentrations of 12% in healthy aortas in contrast to 1% in aortic aneurysms. The concentration of collagen was 85% in healthy aortas and 89% in aortic aneurysms. The wall thickness in the aneurismal aortas was almost the same as in healthy aortas.

U.S. Pat. No. 7,252,834 (titled “Elastin Stabilization of Connective Tissue”) appears to teach the in-situ application of a chemical compound that interacts with the elastin in the tissue to stabilize the connective tissue. The method specifically proposes the application of a phenolic compound to stabilize aortic aneurysms. The inventors argue that preservation of elastin in the aortic tissue provides stability to the vessel wall, and degradation of the elastin leads to dilation of the aorta and ultimately aortic rupture. The Applicant of the current patent application challenges the rationale and effectiveness of the methods and other information described in U.S. Pat. No. 7,252,834, and proposes an alternative method to stabilize aneurismal tissue.

The results in the literature suggest that collagen plays a more dominant role in the stability of aortic tissue than elastin. Dobrin (supra) showed that loss of elastin does not destabilize arterial tissue as long as the collagen structure stays intact. Rizzo (supra) demonstrates that the concentration of elastin can be greatly reduced in aneurismal tissue. Stabilization of the remaining elastin can contribute little to the overall stability. Conversely, collagen was shown to not only prevent dilatation of human arteries but also provide mechanical strength against rupture. During the disease progression, collagen remains present in a high concentration in the aortic wall until synthesis of new collagen cannot keep up with enzymatic degradation of existing collagen ultimately resulting in aneurysm rupture. It is concluded that the proposed cross-linking of elastin as taught in U.S. Pat. No. 7,252,834 does not necessarily stabilize aneurismal tissue. Furthermore, intervention below a dilated aortic diameter of 5 cm is currently not recommended. The content of elastin may already be greatly diminished at that level of dilation.

It is proposed that stabilization of aneurismal tissue should focus on the preservation of collagen. U.S. Provisional Patent Application No. 60/987,268, titled “Method and Agent for In-Situ Stabilization of Vascular Tissue” teaches a novel method of stabilizing vascular tissue by cross-linking collagen with catechins and is incorporated by reference as if fully set forth herein. The methods and agents described therein can readily be applied to aneurysms. Although some of the specific embodiments described herein relates to aortic aneurysms, the methods and apparatus can also be applied to aneurysms in other arteries and other degenerative arterial diseases, such as but not limited to, dissections.

The effectiveness of catechin in cross-linking collagen was demonstrated in a bench top experiment.

Experiment 1:

A 7 cm×7 cm piece of pericardial tissue was fixed in 40 ml of 0.5% EGCG/phosphate buffered saline (PBS), pH 7.42 in a 100 mm petri dish at 37° C., 82 rpm shaking speed, for 15 min. After 15 minutes, the tissue was quickly rinsed with PBS. Three samples were cut out for immediate shrinkage temperature testing (Group A). Shrinkage temperature can be an indication of the stability of the tissue. Increased shrinkage temperature is associated with an increased resistance to degradation. An additional 7 cm×7 cm piece of pericardial tissue was fixed in 40 ml of 0.5% EGCG/PBS, pH 7.42 in a 100 mm petri dish at 37° C. for 48 hours and quickly rinsed in PBS for 15 minutes. Six samples were cut out for shrinkage temperature testing (Group B). Six samples of fresh pericardial tissue were also cut for shrinkage temperature testing (Group C—Control Group).

Observation:

1. Group A samples looked light pink (from opaque white) and more rigid than fresh tissue but still soft. The solution looked dark pink.

2. Group B was dark brown (mocha color), soft and flexible. The PBS solution turned dark yellow.

3. Group A and B demonstrated an increase in the shrinkage temperature over fresh pericardial tissue (Group C).

TABLE 1

| Group | Condition | Shrinkage Temperature (° C.) |
|---|---|---|
| A | Cross-linked for 15 min | 72.2 +/− 0.5 |
| B | Cross-linked for 48 hours | 86.3 +/− 0.6 |
| C | Fresh Tissue | 62.6 +/− 0.6 |

The results in Table 1 demonstrate that Catechin increases the stability of collagen via cross-linking within 15 minutes of application under physiological conditions.

The experiments described in U.S. Provisional Patent Application No. 60/987,268 indicate that application of catechin for only 15 minutes will already demonstrate a stabilizing effect on collagen. At 24 hours, full cross-linking can be accomplished. In some embodiments, the application of catechin in the range of 15 minutes to several hours is therefore proposed.

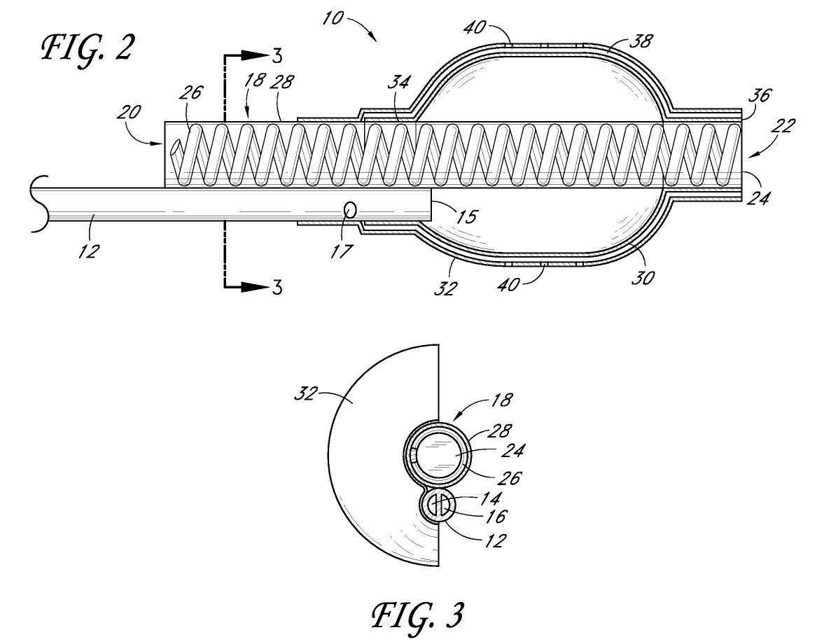

The aforementioned agents can be delivered to the desired arterial location or other suitable location by any suitable method, including but not limited to any of the methods described herein. FIGS. 2-16B describe various embodiments of drug delivery catheters and dilation catheters, which can be used to administer any of the therapeutic agents described herein. One such drug delivery catheter is described in additional detail in U.S. Pat. No. 5,295,962 to Crocker et al.

FIG. 2 is a partial sectional side view of one arrangement of a drug delivery and temporary stent catheter. Referring to FIG. 2, there is disclosed a combination drug delivery and temporary stent catheter. Although the illustrated embodiment can incorporate both the drug delivery and temporary stent features, catheters incorporating only the drug delivery feature or a drug delivery feature in combination with another therapeutic procedure or device can also be readily produced in accordance with the disclosure herein, as will be appreciated by one of skill in the art. In addition, the catheter can readily be used for angioplasty dilatation as well.

The catheter 10 of the illustrated embodiment can comprise an elongate tubular body 12 for extending between a proximal control end (not illustrated) and a distal functional end. Tubular body 12 can be produced in accordance with any of a variety of known techniques for manufacturing balloon tipped catheter bodies, such as by extrusion of appropriate biocompatible plastic materials. Alternatively, at least a portion or all of the length of tubular body 12 can comprise a spring coil, solid walled hypodermic needle tubing, or braided reinforced wall as is well understood in the catheter and guidewire arts.

In general, tubular body 12 has a generally circular cross-sectional configuration having an external diameter within the range of from about 0.030 inches to about 0.065 inches. Alternatively, a generally triangular cross-sectional configuration can also be used, with the maximum base to apex distance also within the range of from about 0.030 inches to about 0.065 inches. Other non circular configurations such as rectangular or oval can also be used. In peripheral vascular applications, the body 12 can have, but is not limited to, an outside diameter within the range of from about 0.039 inches to about 0.065 inches. In coronary vascular applications, the body 12 can have, but is not limited to, an outside diameter within the range of from about 0.030 inches to about 0.045 inches.

Diameters outside of the preferred ranges can also be used, provided that the functional consequences of the diameter are acceptable for a specified intended purpose of the catheter. For example, the lower limit of the diameter for tubular body 12 in a given application will be a function of the number of fluid or other functional lumen contained in the catheter, together with the acceptable flow rate of dilatation fluid or drugs to be delivered through the catheter.

In addition, in some embodiments, the catheter shaft can be configured to have sufficient structural integrity (e.g., “pushability”) to permit the catheter to be advanced to distal arterial locations without buckling or undesirable bending of the catheter body shaft. In some embodiments, the ability of the catheter body shaft to transmit torque can also be desirable, such as in embodiments having a drug delivery capability on less than the entire circumference of the delivery balloon. Larger diameters generally have sufficient internal flow properties and structural integrity, but reduce perfusion in the artery in which the catheter can be placed. In addition, increased diameter catheter bodies tend to exhibit reduced flexibility, which can be disadvantageous in applications requiring placement of the distal end of the catheter in a remote vascular location.

As can best be seen by reference to FIG. 2, the tubular body 12, in accordance with the illustrated embodiment, can comprise at least a first lumen 14 and a second lumen 16 extending axially therethrough. Inflation lumen 14 can be in fluid communication with the interior of inflation balloon 30 by way of port 15. Drug delivery lumen 16 can be in fluid communication with a drug delivery balloon 32 by way of port 17. In this manner, inflation fluid or fluid medication can be selectively introduced into the inflation balloon 30 and drug delivery balloon 32, as will be described in greater detail below.

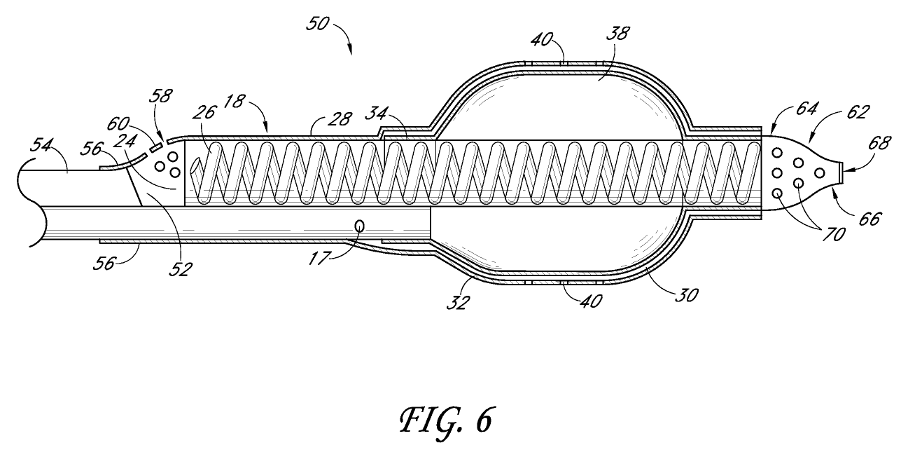

Additional lumen can readily be formed in tubular body 12 by techniques known in the art. In some embodiments (not illustrated), a third lumen can be provided having an opening at its proximal end and a closed distal end. This third lumen can receive a wire to improve pushability of the catheter. A further embodiment, illustrated in FIG. 6 and discussed infra, can be provided with a guidewire lumen for over-the-wire manipulation.

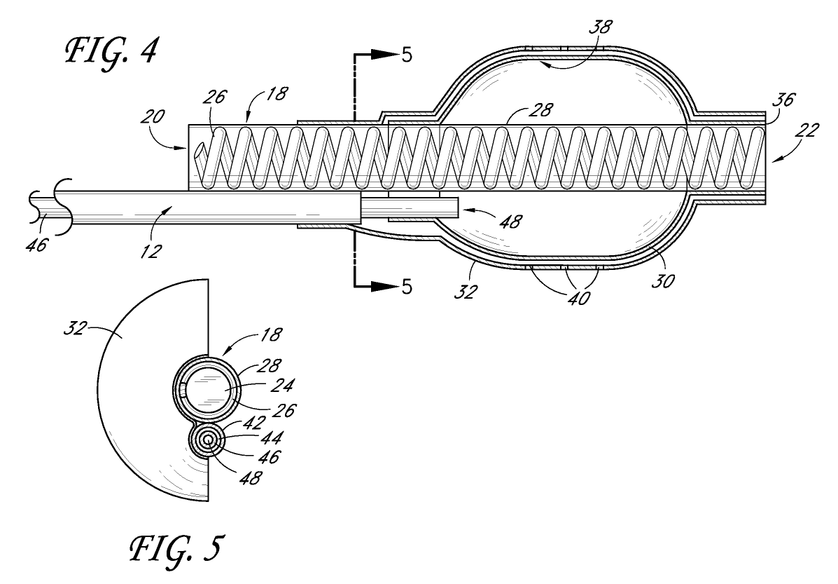

FIG. 4 is a partial sectional side view of another arrangement of a catheter, having a coaxially configured catheter body. FIG. 5 is a cross-sectional view taken along the lines 5-5 in FIG. 4. With reference to FIGS. 4 and 5, in a modified embodiment of the catheter body, two or more lumens are disposed in a concentric arrangement. Tubular body 12 can comprise an outer tubular wall 42 defining a first lumen 44 for communicating a fluid to the distal end of the catheter. An inner tubular wall 46 defines a second lumen 48. In the illustrated embodiment, inner lumen 48 is in fluid communication with the inflation balloon 30, and outer lumen 44 is in fluid communication with the drug delivery balloon 32. Concentric lumen catheter bodies can be manufactured in accordance with techniques known in the art.

A temporary stent 18 can be secured to the distal end of tubular body 12. As illustrated in FIG. 2, the longitudinal axis of temporary stent 18 can be laterally displaced from the longitudinal axis of tubular body 12. As illustrated in FIG. 3, stent 18 can comprise a first end 20, a second end 22 and a lumen 24 extending therebetween. Blood flow through lumen 24 can occur in either direction, depending upon the location of percutaneous insertion and the direction of transluminal travel of the catheter.

In general, it is desired that the ratio of the interior cross-sectional area of lumen 24 to the maximum exterior cross-sectional area of the deflated balloon be maximized in order to optimize perfusion across the inflation balloon 30 while inflation balloon 30 is inflated. Catheters arrangements having a perfusion deflated profile of 0.055 inches or greater can be produced having an interior lumen 24 with an interior diameter of at least about 0.030 inches, and in another arrangement about 0.039 inches or greater. This fits readily within the lumen of a guide catheter, which can have an internal diameter of about 0.072 inches. Alternatively, the diameter of lumen 24 can be reduced to as low as about 0.012 inches and still function as a guidewire conduit.

In some embodiments, the interior diameter of lumen 24 can be about 0.039 inches (1 mm). This lumen will typically provide a flow at 80 mm Hg of greater than 60 ml/minute. The coil wall thickness of about 0.002 inches adds 0.004 inches to the diameter of stent 18. The outer sheath 28, described infra, has a thickness of about 0.001 inches and can produce an assembled stent 18 having an outside diameter of about 0.045 inches.

The illustrated design can provide a significant passageway 24 cross-sectional area compared to the overall cross-sectional area of stent 18. In this configuration, only the stent 18 and balloon will typically traverse the stenotic site which can be advantageous. The distal end of catheter body 12 (i.e., port 15) typically ends proximally of the stenosis in the preferred application. This parameter is conveniently expressed in terms of the percentage of the outside diameter of stent 18 that the thickness of a single wall of stent 18 represents. In other words, in a preferred embodiment, a 0.003 inch wall thickness is about 6.7% of the 0.045 inch outside diameter.

In some embodiments, this percentage is less than about 14%, and in another less than about 8%, and in another arrangement less than about 5% to optimize perfusion through the inflated balloon. Lower percentages can be achieved through the use of existing or new materials, techniques, or configurations. For example, lower percentages can be obtained by sacrificing pushability or by development or use of new high strength materials. For example, if sufficiently structurally sound for a given application, use of a 0.002 inch stent wall in a 0.045 inch diameter catheter will produce a 4.4% value. In addition, the percentage can be reduced by increasing the outside diameter of the stent to the maximum permitted for a given application.

Temporary stent 18 can comprise a support structure for resisting radial compression of passageway 24 by the inflated balloon 30. Suitable support structures include, but are not limited to, braided or woven polymeric or metal reinforcement filaments or a spring coil 26. Spring coil 26 can comprise a material having suitable biocompatibility and physical properties, such as a stainless steel or platinum wire. Alternatively, polymeric materials such as nylon or Kevlar (DuPont) can also be used. In some embodiments, rectangular ribbon can be used, having cross-sectional dimensions on the order of about 0.001 inches by about 0.003 inches for small vessels, and on the order of about 0.005 inches by about 0.010 inches for use in larger vessels. The wire or ribbon can be wound to produce a coil having an interior diameter within the range of from about 0.030 inches (coronary) to about 0.100 inches (periphery) and an exterior diameter within the range of from about 0.032 inches (coronary) to about 0.110 inches (periphery).

Spring coil 26 can be either “tightly wound” so that adjacent loops of coils are normally in contact with each other, or “loosely wound,” as illustrated in FIG. 2, in which the adjacent loops of coil are normally separated from one another. The selection of a tightly wound or loosely wound coil for use in the present arrangement will be influenced by such factors as the desired weight of the finished catheter, the relative flexibility of the catheter in the region of temporary stent 18, and the amount of radially inwardly directed compressive force exerted by the inflation balloon 30, as will be apparent to one of skill in the art. Radiopacity can also be a factor.

A spring coil 26 can be provided with an outer sheath or coating 28. Sheath 28 can be produced by dipping, spraying, heat shrinking, extrusion, or any other suitable techniques, and can comprise a relatively flexible material having sufficient biocompatibility to enable its use in contact with the vascular intima. Suitable materials for sheath 28 comprise, but are not limited to, linear low density polyethylene such as that produced by Dow, polyethylene terephthalate, nylons, polyester or other known or later developed medical grade polymers.

Inflation balloon 30 can comprise a proximal neck portion 34, a distal neck portion 36, and an intermediate dilatation portion 38. Referring to FIGS. 2 and 4, it can be seen that the proximal neck of each balloon is larger in diameter than the distal neck to accommodate the catheter body 12. Proximal neck portion 34 can be tightly secured to the temporary stent 18 and distal portion of tubular body 12, such as by the use of conventional adhesives, thermal bonding or heat shrinking techniques. The interstitial space formed by the diverging walls of tubular body 12 and temporary stent 18 (in a circular cross-section embodiment) can be provided with a fluid-tight seal such as by filling with adhesive. In this manner, a fluid-tight seal between the proximal neck portion 34 and the elongate tubular body 12 and temporary stent 18 can be provided.

The distal neck 36 of inflation balloon 30 can be provided with a fluid-tight seal with the distal portion of temporary stent 18. This seal can also be accomplished in any of a variety of manners known in the art, such as by the use of heat shrink materials, adhesives, or other thermal bonding or solvent bonding techniques. A distal neck 36 of inflation balloon 30 can, in some embodiments, be heat shrunk onto stent 18. As will be appreciated by one of skill in the art, the sheath 28 can cooperate with the dilatation portion 38 of the inflation balloon 30 to provide a sealed compartment for retaining a dilatation fluid therein.

In some arrangements, the inflation balloon can comprise a relatively non-elastic material such as linear low density polyethylene, polyethyleneterephthalate, nylon, polyester, or any of a variety of other medical grade polymers known for this use in the art. In some embodiments, the geometry, material and seals of balloon 30 can withstand an internal pressure of at least about 5 ATM and, in other arrangements, about 10 ATM without any leakage or rupture. The balloon can be premolded to have an inflated diameter in a catheter intended for peripheral vascular applications within the range of from about 1.5 mm to about 8 mm. The balloon 30 (in a catheter intended for coronary vascular applications) can have an inflated diameter range of from about 1.5 mm to about 4 mm.

Although the illustrated embodiment has been described in terms of an “inflation” balloon 30, it is to be understood that the balloon 30 can also function as a dilatation balloon, such as is well known in the art of percutaneous transluminal coronary angioplasty and other applications in which dilatation of a stenotic region in a body lumen is desired. In an embodiment in which dilatation properties are desired, conventional dilatation balloon materials and design considerations can readily be incorporated, as will be understood by one of skill in the art. Alternatively, if the inflation balloon 30 is merely desired to provide sufficient radially expansive force to press the drug delivery balloon 32 against the wall of the vessel, considerations appropriate for a lower pressure system can be utilized.

The drug delivery balloon 32 can be disposed radially outwardly from the inflation balloon 30. Drug delivery balloon 32 can comprise a generally non-elastic material such as is conventional for angioplasty dilatation balloons, or can comprise an elastic material such as latex or urethane, or any other suitably biocompatible elastomer. Use of an elastic material for drug delivery balloon 32 can assist in reducing the relatively rough edges of the collapsed inflation balloon 30, and thereby reduce trauma to the vascular intima during insertion and withdrawal of the catheter.

Drug delivery balloon 32 can be provided with a plurality of delivery ports 40. Delivery ports 40 can be disposed radially symmetrically about the outer periphery of the delivery balloon 32, or can be limited to only portions of the exterior surface of the delivery balloon 32, depending upon the desired drug delivery pattern. For example, delivery ports 40 can be positioned only on one hemisphere of balloon 32. In another arrangement, delivery ports 40 can extend for less than the entire length of the balloon.

Delivery balloon 32 in a modified embodiment can comprise a material which is inherently permeable and/or porous, without the provision of discrete delivery ports 40. For example, woven or braided filaments or fabrics can be used. For relatively low delivery rate applications, fluid permeable membranes can also be used. In some embodiments, the balloon 32 can be selectively permeable and/or porous, for example, made porous by the application of a release agent.

As can be seen with reference to FIG. 2, drug or other fluid introduced by way of lumen 16 can be expressed by way of port 17 into the interior space of drug delivery balloon 32. The inflated volume of inflation balloon 30 can cause the drug to be expelled by way of ports 40 outside of the drug delivery system.

In some embodiments, the relative inflated dimensions of the delivery balloon 32 and the inflation balloon 30 are such that a minimum amount of drug is retained between the two balloons. Thus, the inflated inflation balloon 30 can substantially completely fill the interior chamber of drug delivery balloon 32 to efficiently expel essentially all of the fluid introduced into drug delivery balloon 32 by way of drug delivery lumen 16. Residual volume of drugs contained in lumen 16 can be expelled outside of the balloon such as by following the drug with a small volume of normal saline or other “rinse” solution, as will be understood by one of skill in the art.

In further arrangements, the inflation and drug delivery can be accomplished by the same balloon. The permeability rate of the balloon material, or the diameter and number of delivery ports 40 can be sufficiently small that the balloon is sufficiently firmly inflated without delivery at an excessive rate. Appropriate permeability rates for the balloon material can be determined through routine experimentation, in view of such factors as the viscosity of the drug, desired delivery rate and the desired radially expansive force to be exerted by the balloon.

Referring to FIG. 6, there is disclosed an over-the-wire embodiment of the delivery device. Over-the-wire catheter 50 can be provided with a third lumen 52 extending through housing 54. In some embodiments, housing 54 can comprise a separate tube which can be secured along the outside of catheter body 12 such as by adhesives or other plastic bonding techniques known in the art. In another arrangement, however, housing 54 can comprise an integrally formed three lumen catheter body as is well known in the art. Lumen 52 can be provided with a sufficient interior cross-sectional area to axially slidably receive a conventional guidewire, such as a 0.014 inch guidewire.

In some embodiments, an extruded three lumen catheter body can be prepared in accordance with techniques known in the art. One lumen, intended as guidewire lumen 52, has an internal diameter of at least about 0.016 inches. The wall surrounding lumen 52 can be thereafter cut down using conventional cutting or grinding equipment. Alternatively, the catheter body can be integrally molded with one lumen shorter than the other two, such as by injection molding about removable wire mandrels, and post molding cutting steps.

The distance between the distal end of lumen 52 and the proximal end of stent 18 can range from essentially zero up to an inch or more, particularly if a cover 60 is used as described infra. In some embodiments, the distance between the distal end of lumen 52 and the proximal end of stent 18 can be no more than about 12 inches, and in another arrangement no more than about 0.2 inches. In the arrangement illustrated in FIG. 6, the distal end of lumen 52 can be about 0.08 inches from the proximal end of stent 18, and about 0.5 inches from port 15.

In some embodiments, a distal extension of the longitudinal axis of lumen 52 can be aligned to extend through the lumen 24 in temporary stent 18. In this manner, a guidewire which can be threaded distally through lumen 52 can thereafter be directed through lumen 24. This design facilitates removal and reinstallation of the guidewire while the catheter 50 is in place.

In some embodiments, the proximal neck of one or both of the balloons 30, 32 can extend in a proximal direction to form a seal 56 around housing 54. In this manner, a cover 60 can be provided for the proximal end of lumen 24. Cover 60 can both assist in the withdrawal of the catheter from the vascular system, as well as assist in ensuring that a guidewire advanced distally through lumen 52 can be guided into lumen 24. In embodiments incorporating this feature, the cover 60 can be provided with a plurality of perfusion ports 58 to permit continued perfusion through cover 60 and lumen 24. In some embodiments, the cover 60 can comprise a proximal extension of delivery balloon 32.

As an additional optional feature of certain arrangements, there can be provided a flexible, generally cone-shaped distal tip 62 for facilitating distal advancement of the catheter 50 along a previously positioned guidewire (not illustrated). Distal tip 62 can comprise a relatively large diameter proximal portion 64 which can be an integral extension of either inflation balloon 30 or delivery balloon 32. Tip 62 can taper radially inwardly in a distal direction to a relatively narrow portion 66 having an axially-aligned guidewire and perfusion opening 68 therein.

The axial length of distal tip 62 can be varied depending upon a variety of factors such as the diameter and rigidly of the material used. In some arrangements, distal tip 62 can be made from the same material as delivery balloon 32, and can be formed by axially stretching the distal end of balloon 32 with the application of heat. The proximal port diameter can be about 0.035 to 0.050 inches and the distal opening 68 in some embodiments has a diameter of about 0.016 inches. The axial length of tip 62 can be about 0.4 inches.

To optimize perfusion through lumen 24, a plurality of ports 70 can be distributed about the periphery of distal tip 62. Ports 70 in some embodiments can have a diameter of at least about 0.030 inches, and generally as many ports 70 (and ports 58) as possible can be provided without unduly interfering with the structural integrity of the tip 62 (or cover 60). The precise configuration of distal tip 62 can be varied considerably, while still performing the function of providing a guide for the guidewire and permitting optimum perfusion through lumen 24.

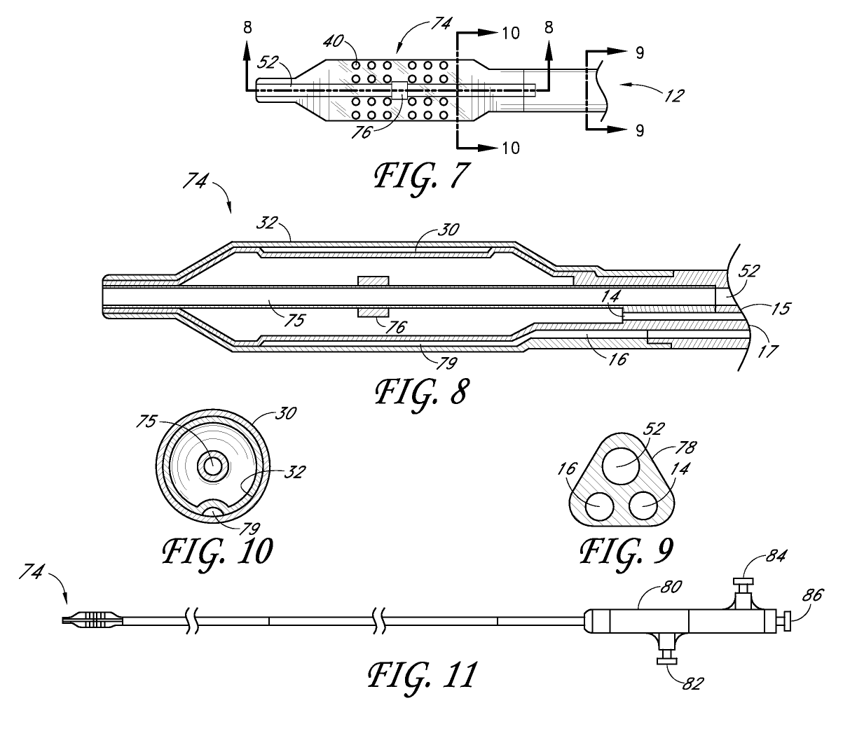

FIG. 7 is a partial sectional side view of a non-stent arrangement of a catheter. FIG. 8 is a cross-sectional view taken along the lines 8-8 in FIG. 7. FIG. 9 is a cross-sectional view taken along the lines 9-9 in FIG. 7. FIG. 10 is a cross-sectional view taken along the lines 10-10 in FIG. 7. FIG. 11 is a side view of a non-stent arrangement in communication with a fluid delivery and guide-wire entry apparatus. Referring to FIGS. 7-11, there is disclosed a nonperfusion catheter embodiment 74 which, in some embodiments, also does not include a temporary stent. The non-perfusion embodiment 74 can be designed for use in percutaneous coronary transluminal angioplasty and adjunctive site specific intraluminal infusion of pharmacological agents.

The non-perfusion embodiment 74 can comprise a tubular body 12 which includes an inflation lumen 14, a drug delivery lumen 16, and a guidewire lumen 52. Two concentric balloons, an inner inflation balloon 30, and an outer delivery balloon 32 can be connected to the tubular body 12. Alternatively, the inflation balloon and delivery balloon can be disposed on opposing sides of the longitudinal axis of the body 12, such as for delivery of medication to an eccentric delivery site.

The inflation lumen 14 can be in fluid communication with the inflation balloon 30 through port 15, the delivery lumen 16 can be in fluid communication with the drug delivery balloon 32 through port 17, and the guidewire lumen 52 can be in communication with a central lumen 75 which can allow a guidewire to pass through the distal end of the catheter. A radiopaque marker 76 can be placed around the central lumen 75 in the center of the inflation balloon 32 to assist in positioning the catheter in the desired location. The tubular body 12 can be an integrally formed three lumen catheter body 78 as is well known in the art.

In the illustrated arrangement, the three lumen catheter body 78 can have a triangular cross-section for a majority of the length of the tubular body 12, as illustrated in FIG. 9. The triangular shape of the tubular body 12 provides a clearer fluoroscopy picture of the tubular body 12 within the patient, as the tubular shape can reduce the cross-sectional area of the tubular body 12 by up to 30%. The reduction in cross-sectional area of the tubular body 12 thus allows for the injection of up to 30% more dye into the guiding tube (not shown) which can provide a clearer fluoroscopy picture of the tubular body within the patient. Further, the reduction in cross-sectional area of the tubular body 12 can allow for more perfusion to occur around the catheter body 12.

In the illustrated embodiment, a distal extension of the longitudinal axis of the guide wire lumen 52 can be aligned with a central lumen 75. In this manner, a guidewire which can be threaded distally through lumen 52 can thereafter be directed through lumen 75. This design can facilitate removal and reinstallation of the guidewire while the catheter 74 is in place.

As illustrated in FIG. 10, the central lumen 75 is typically concentric with both the inflation balloon 30 and delivery balloon 32 and extends through the center of the inflation balloon 30 and exits out the distal end of the catheter. The delivery lumen 16 extends into the catheter body and can be in fluid communication with the delivery balloon 32. As described infra, during infusion of a fluid into the delivery balloon a small luminal channel 79 can be maintained between the inflation and delivery balloons 30, 32 to enable the flow of the fluid to the delivery ports 40. The inflation lumen 14 terminates at the proximal end of the catheter body and is therefore not shown in FIG. 9.

The inflation and delivery balloons 30, 32 can be between 2.0 cm and 6.0 cm in length. However, balloon length can be varied depending upon the requirements of a particular desired application. The deflated profile of the inflation and delivery balloons 30, 32 can be between 0.025 inches and 0.070 inches in diameter. The inflation balloon 30 and delivery balloon 32 can be sealed, using a process which will be described infra, such that a portion of the distal ends and a portion of the proximal ends of the balloons can be sealed together.

The delivery balloon 32 can include a series of discrete delivery ports 40 to enable the delivery of the infused liquid to the desired location. The delivery ports can be between 100 μm and 300 μm, and in other arrangements can be about 250 μm in diameter. The discrete delivery ports 40 can be disposed radially symmetrically about the outer periphery of the delivery balloon 32 and cover the mid section of the balloon. Depending on the size of the delivery balloon 32 there can be 3-50 delivery ports in the delivery balloon 32. Alternatively, fewer delivery ports 40 can be used and disposed only on one hemisphere of the balloon or only the distal end of the balloon, depending on the desired drug delivery pattern.

In the non-perfusion embodiment, due to the relatively large diameter of the delivery ports 40 and the large number of ports 40 on the catheter, the drug can slowly drip or “weep” out of the ports 40. The large number of the large sized delivery ports 40 and the initial low pressure which can be used to infuse the drug into the catheter opening results in a very low outlet pressure at the ports 40 of the catheter tip and therefore causes the drug to “weep” out of the ports 40 rather than exiting under a high pressure flow. The “weeping” action causes the drug to exit the catheter tip at a site specific location, however the low pressure delivery of the drug is not enough to penetrate the arterial wall beyond the elastic lamina layer. The delivery of the drug to the artery while maintaining the structural integrity without the penetration of the drug past the luminal wall of the artery will herein be referred to as intraluminal drug delivery, i.e., within the arterial lumen. Further, depending on the use of the catheter, i.e., for PTCA dilatation, for drug delivery or for both operations, the level of inflation of the inflation balloon 30 will influence the drug delivery rate as described infra.

In another embodiment of the non-perfusion catheter, the size of the delivery ports 40 can be reduced to reduce the “weeping” effect and enable a steady flow of the drug to be delivered to the desired vascular site. In a further embodiment, the size of the delivery ports 40 remain the same size as described above and the drug delivery pressure is increased to provide a steady flow of the drug to the desired vascular location. Generally, the total cross-sectional area of all ports can be at least 300% greater and no more than 400% greater than the cross-sectional area of the delivery lumen 16. In a one embodiment, the total area of the delivery ports 40 and the pressure of the fluid which can be delivered to the vascular site are both varied to achieve the desired delivery profile to the vascular site.

In yet another arrangement, of the non-perfusion catheter, the delivery balloon 32 can comprise a material which can be inherently permeable and/or porous, without the provision of discrete delivery ports 40. For example, woven or braided filaments or fabrics can be used. For relatively low delivery rate applications, fluid permeable membranes can also be used. In some embodiments, the balloon 32 can be selectively permeable and/or porous, for example, made porous by the application of a release agent.

Drug delivery using the non-perfusion embodiment 74 can be performed alone or in combination with a conventional PTCA procedure. When used in combination with a conventional PTCA dilatation operation, the drug can be delivered before, during or after the PTCA procedure. In some embodiments, the non-perfusion embodiment 74 will be used to deliver thrombolytic agents, such as urokinase, t-PA and the like, when indicated.

When drug delivery is performed before or after conventional PTCA, the inner inflation balloon 30 can be inflated or deflated to a relatively low pressure, such as between about 0.4 ATM-1.5 ATM, preferable to about 0.5 ATM. A small luminal channel 79 (with reference to FIG. 10) can be maintained between the inner inflation balloon 30 and the outer delivery balloon 32. The luminal channel 79 is typically on the order of approximately 0.01 inches in diameter when the inflation balloon 30 is inflated to a constant 0.5 ATM. Channel 79 can permit communication of the drug from delivery lumen 16 to the outer ports 40 in the delivery balloon 32 at an even and continuous rate. As the pressure applied to the drug delivery balloon 32 increases the flow rate out of the ports 40 increases. However, the risk of a sufficiently high pressure to perforate the vascular wall can be minimized by appropriate sizing of the channel 79 with respect to the total cross-sectional area of the ports 40 as will be readily understood by one skilled in the art. Drug delivery before the PTCA dilatation can be advantageous as any thrombus which is located near the area to be treated can be dissolved before dilation.

When the inner inflation balloon 30 is inflated to between 2 ATM and 12 ATM, the catheter can be used for dilatation of a stenosis using conventional PTCA techniques. During the PTCA procedure, a drug can also be introduced into the delivery balloon 32 and delivered through the ports 40 to the specific location on the arterial wall. Even during the PTCA procedure, the resultant pressure within the delivery balloon 32 is not enough to deposit the drug into the laminal layer of the arterial wall. Drug delivery during a PTCA procedure can be advantageous to assist in treating the stenosis while the dilatation is occurring. After the PTCA procedure is complete if additional thrombus is discovered, the catheter can be used to deliver medication to the newly discovered thrombus.

Once the drug delivery and or PTCA procedure is complete and the catheter is prepared for extraction from the artery, the pressure is first reduced at the outer delivery balloon 32 to halt continual infusion of the drug during extraction. However, the outer delivery balloon 32 will not immediately collapse. Next, the pressure in the inner inflation balloon 30 is reduced such as by aspiration with the inflation syringe, causing the inner balloon 30 to deflate. The inner and outer balloons 30, 32 can be sealed together at both axial ends, as described below, thus the reduction in diameter of the inner balloon 30 reduces the profile of the outer balloon 32.

In an embodiment, at least a portion of the inflation balloon 30 can be connected to at least a portion of the delivery balloon 32. This structure permits the inflation balloon to “pull” the delivery balloon with it when the inflation balloon is being aspirated to minimize the external dimensions. The connection between the inflation balloon 30 and delivery balloon 32 can be accomplished in any of a variety of techniques as will be understood by one of ordinary skill in the art.

To provide a relatively small delivery site, the inflation balloon 30 and drug delivery 32 balloon can be heat sealed together along almost the entire axial length of the balloon, leaving only a relatively small unsealed area to allow the delivery of the desired drug. To provide a relatively large delivery site, while maintaining the advantage of “pulling” the delivery balloon 32 in with the inner inflation balloon 30, only the very ends of the inflation balloon 30 and delivery balloon 32 can be sealed together. In addition, as the diameter of the delivery ports 40 increases, the percentage of the axial length of the two balloons 30, 32 that is sealed together must necessarily increases to enable the outer delivery balloon 32 to be “pulled” in by the aspiration of the inner balloon 32, as will be understood by one skilled in the art. Further, as the overall pressure used to aspirate the inner balloon decreases, the percentage of the axial length of the two balloons 30, 32 that is sealed together must also be increased, as will be understood by one skilled in the art.

In some embodiments, about 25% of the total axial length of the inflation balloon 30 can be sealed to the delivery balloon 32 at the proximal end and about 25% of the total axial length of the inflation balloon 30 can be sealed to the delivery balloon 32 at the distal end to aid in the deflation process as described above. Desirably, the entire circumference of the distal ends of the inflation 30 and delivery balloons 32 can be sealed together. A relatively large percentage of the proximal ends of the inflation balloon 30 and delivery balloon 32 can be sealed together. The small portion of the two balloons 30, 32 on the proximal end that is not sealed together can form the very small luminal channel 79 between the inflation balloon 30 and the delivery balloon 32.

FIG. 11 illustrates the non-perfusion embodiment 74 of the catheter in communication with a fluid delivery and guidewire entry apparatus 80. An inflation port 82 can be provided for the delivery of the inflation fluid to the inflation lumen 14. A delivery port 84 can be provided for delivery of the infusion fluid to the delivery lumen 16. Port 86 permits entry of a guidewire into the guidewire lumen 52. The guidewire entry port 86 can be positioned along the longitudinal axis of the catheter to easily align the guidewire with the guidewire lumen 52 to prevent any unnecessary bending of the guidewire during insertion into the lumen 52. The fluid delivery and guide-wire entry apparatus 80 can remain outside the patient so the doctor can control the delivery of the fluid and the guidewire from outside the patient's body. In an alternate embodiment, an indeflator (not shown), which can be basically a syringe connected to a pressure reading device, can be attached to the inflation and delivery ports 82, 84 to monitor the pressure of the fluid which can be delivered to the inflation and delivery balloons 30, 32.

The catheters incorporating various features discussed above can be manufactured in a variety of ways. Some of the preferred manufacturing techniques for catheters described herein are discussed below. For example, the perfusion conduit or temporary stent 18 assembly can be manufactured by winding a coil of suitable spring wire, typically having a diameter or thickness dimension in the radial direction of the finished spring of about 0.002 inches. The wire can be wound about a mandrel sufficient to produce a spring having a lumen 24 with a diameter of about 0.039 inches.

The coil can be provided with an outer sheath or coating, as has previously been discussed. In some embodiments, the tightly coiled wire can be held securely about the mandrel such as by clamping or soldering each end to the mandrel so that the coil is not permitted to unwind slightly and expand radially following release as will be understood by one of skill in the art. The tightly wound coil is thereafter inserted within a tubular sleeve, such as an extruded non-cross-linked polyethylene tubing of desired size. The spring coil is then released from the mandrel, so that the spring unwinds slightly within the polyethylene tube to produce a tight fit.

Typically, the minimum wall thickness of extruded polyethylene tubing as discussed above can be no less than about 0.002 inches. This wall thickness can be reduced by heat stretching the polyethylene tubing either prior to insertion of the spring or directly onto the pre-wound spring coil to provide a tight seal. The heat stretching step has been determined to produce a polyethylene coating on the spring coil having a wall thickness as low as about 0.001 inches. Thus, the overall diameter of the stent 18 assembly can be reduced by about 0.002 inches.

The body of the catheter can be separately produced, typically by a combination of extrusion and post-extrusion processing steps. For example, an elongate triple lumen triangular cross-section catheter body can be produced by extrusion of high density polyethylene, to produce a body having a minimum wall thickness within the range of from about 0.003 to about 0.005 inches.

To minimize the overall cross-sectional area of the assembled catheter, the distal portion of the tubular body 12 can be reduced in diameter and wall thickness such as by axially stretching under the influence of heat. Stretching can be accomplished by inserting, in a preferred embodiment, a 0.016 inch diameter pin in the guidewire lumen 52, and a 0.010, inch diameter pin in each of the inflation lumen 14 and drug delivery lumen 16. The distal end of the catheter body can be thereafter heat stretched nearly to the limit before breaking. The result of the stretching reduces the cross-section of the triangular catheter body, from base to apex, from about 0.039 inches in the unstretched condition to about 0.025 inches following heat stretching.

The transition zone between the unstretched catheter body 12 and the distal axially stretched portion can occur within about 0.01 inches proximally of the proximal end of the temporary stent 18 in the assembled catheter. It has been determined by the present inventor that the decrease in structural strength of the heat stretched catheter body does not appear to adversely impact the integrity of the assembled catheter, at least in the designs disclosed herein.

The inflation balloon 30 and drug delivery balloon can be manufactured in any of a variety of manners which are now conventional in the art, such as free-blowing polyethylene, polyethylene terephthalate, nylon, polyester, or any of a variety of other medical grade polymers known for this use. Generally, the interior inflation balloon 30 can be produced by blowing relatively long sections of cross-linked polyethylene within a mold to control the outside diameter. The use of cross-linked polyethylene facilitates heat sealing to the coil, which can be coated with non-cross-linked polyethylene.

The sections of inflation balloon material can be thereafter heat stretched at the proximal and distal necks of a balloon down to a thickness of about 0.001 inches and a diameter which relatively closely fits the portion of the catheter body to which it is to be sealed. The appropriate length can be cut, depending upon the desired length of the balloon and balloon necks in the finished catheter.

The proximal neck can be heat sealed around the catheter body 12 and the temporary stent 18 as illustrated in FIGS. 2 and 6. In general, the length of the proximal and distal neck which can be secured to the catheter body can be within the range of from about 0.05 inches to about 0.1 inch, except in an embodiment such as illustrated in FIG. 5, in which the proximal and distal balloon necks can be as long as necessary to accomplish their functions as a proximal cover or distal tip. The distal end of the inflation balloon 30 can be thereafter heat sealed around the distal end of the temporary stent 18.

The outer balloon can thereafter be assembled in a similar manner, following “necking down” of the axial ends of the balloon by axial stretching under the application of heat. In an embodiment utilizing cross-linked polyethylene for the outer delivery balloon, the delivery balloon is typically secured to the axial ends of the inflation balloon through the use of a UV-curable adhesive, due to the difficulty in thermally bonding cross-linked polyethylene to cross-linked polyethylene.

However, it is to be understood that the material utilized for the outer delivery “balloon” can be varied considerably, and the term “balloon” as used in the context of the delivery balloon is intended to be only generally descriptive of this structure. For example, in addition to perforated balloons, a wide variety of materials not conventionally used for true balloons can also be used. Woven or braided fibers such as dacron, or fluid permeable membranes can desirably be used for the outer delivery balloon, as has been discussed.

In another arrangement, the cross-sectional configuration of the temporary stent 18 can change from substantially circular at the distal end thereof to substantially rectangular or square at the proximal end thereof. This configuration can be accomplished by winding the spring coil around a mandrel having a square cross-sectional portion, a transition portion, and a round cross-sectional portion. The transition portion on the resulting spring can be located in the assembled catheter at about the line 5-5 on FIG. 4. This allows the temporary stent portion 18 to retain the same internal cross-sectional area, while reducing the maximum width of the assembled catheter.

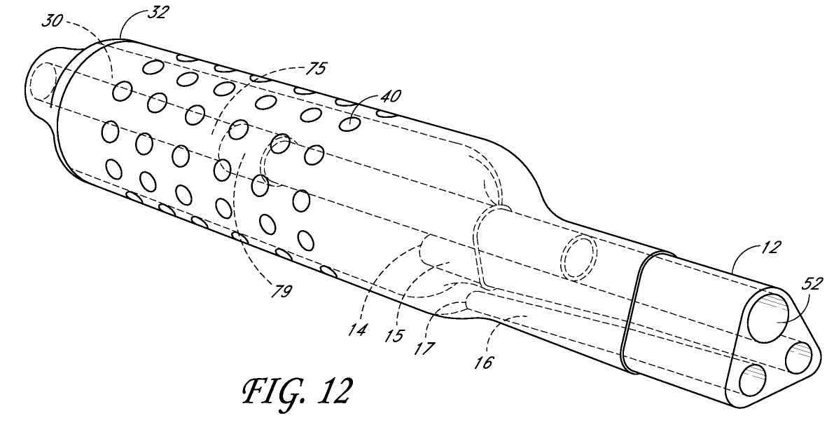

In the non-perfusion embodiment 74, the distal end of the catheter body 12 can be cut away to separately expose each of the three lumen as illustrated in FIG. 12. First, a small portion of the catheter body can be cut away to expose the drug delivery lumen 16. Next, a larger length can be cut away to expose the inflation lumen 14. Finally, an additional portion can be cut away to expose the guidewire lumen 52. The central lumen 75 abuts the guidewire lumen and the two lumen can be joined together using an adhesive or any other suitable bonding process. A radio opaque marker 76 can be positioned in the center of the catheter 74 concentric to the central lumen 75.

A long steel mandrel can be inserted into each of the inflation lumen 14, delivery lumen 16, and the guidewire lumen 52 which extends through the central lumen 75, the mandrels extending along the entire length of the catheter body 12. The steel mandrels can be provided to keep the lumen from sealing closed during the balloon assembly procedure. The inflation balloon 30 can be placed over the central lumen 75 and the inflation lumen 14. The inflation balloon 30 can be then bonded to the central lumen 75 and the inflation lumen 14 at the proximal end and to the central lumen 75 at the distal end. The inflation balloon 30 can be bonded to the inflation lumen 14 and the central lumen 75 using any of a variety of bonding techniques known to those skilled in the art, such as solvent bonding, thermal adhesive bonding, or by heat sealing. In some embodiments, the inflation balloon 30 can be heat sealed to the inflation lumen 14 and the central lumen 75.

The delivery balloon 32 can be bonded to the catheter body 12 by any of a variety of bonding techniques such as solvent bonding, thermal adhesive bonding or by heat sealing depending on the type of balloon material used. In the present arrangement, cross-linked polyethylene balloons can be used, therefore the inflation 30 and delivery balloons 32 can be heat sealed together as follows. The wire mandrel can be removed from the central lumen 75 and guidewire lumen 52 and a 0.01 inch diameter teflon rod can be placed in the central lumen 75 to insure that the central lumen 75 is not sealed closed during the assembly process.

The delivery balloon 32 can be positioned at the proximal end of the catheter 74 to cover the inflation balloon 30 and the delivery lumen 16. To create the luminal channel 79, a teflon rod of a diameter which can be the same as the desired diameter of the luminal channel 79 can be placed between the inflation balloon 30 and the deliver balloon 32 at the proximal end of the two balloons 30, 32. A teflon capture tube (not shown) can be positioned over the delivery balloon 32 and covers the portion of the proximal end of the delivery balloon 32 which is to be sealed to the inflation balloon 30. The teflon capture tube can be a generally tubular body which has approximately the same diameter as the inflated diameter of the inflation balloon 30 and can be made of teflon. The inflation balloon 30 can be inflated to a pressure which is sufficient to force the delivery balloon 32 against the wall of the teflon capture tube. The inflation balloon 30 can be inflated to about 30-50 psi. The capture tube can be heated by any of a number of heating means such as electric coils or a furnace to a temperature which is sufficient to bond the two balloons 30, 32 together. In this case, the cross-linked polyethylene balloons can be heated to a temperature of about 300° F., which can cause both balloons to seal together. The teflon capture tube can be then cooled to a temperature below the melting temperature of the two balloons 30, 32. The inflation balloon 30 can be deflated and the catheter can be removed from the capture tube. The teflon rod used to create the luminal channel 79 can be removed.

To seal the distal end of the delivery balloon 32 to the inflation balloon 30, the delivery balloon can be positioned at the distal end of the catheter 74 and completely cover the inflation balloon 30. The teflon capture tube (not shown) can be positioned over the delivery balloon 32 and covers the portion of the distal end of the delivery balloon 32 which is to be sealed to the inflation balloon 30. The inflation balloon 30 can be inflated to force the delivery balloon 32 against the wall of the teflon capture tube. The inflation balloon 30 can be inflated to about 30-50 psi. As above, the capture tube can be heated by any of a number of heating means such as electric coils or a furnace to a temperature which is sufficient to bond the two balloons 30, 32 together. In this case, the cross-linked polyethylene balloons can be heated to a temperature of about 300° F., which can cause both balloons to seal together. The teflon capture tube can be then cooled to a temperature below the melting temperature of the two balloons 30, 32. The inflation balloon 30 can be deflated and the catheter can be removed from the capture tube. The teflon rod can be removed through the distal end of the central lumen 75. The steel mandrels can be removed from the inflation lumen 14 and the delivery lumen 16 through the proximal end of the catheter body 12.

In some embodiments, a site can be identified in a body lumen where it is desired to deliver an amount of a medication or other gas or fluid. For example, thrombolytic or restenosis inhibiting drugs can be desirably introduced directly to the affected wall following dilatation. Alternatively, anticoagulants, plaque softening agents or other drugs can desirably be delivered directly to the site of a thrombosis or other vascular anomaly. A conventional angioplasty guidewire can be percutaneously transluminally inserted and advanced to the desired treatment site. Guidewires suitable for this purpose are commercially available, having a variety of diameters such as 0.014 inches.

The distal end 22 of temporary stent 18 can be threaded over the proximal end of the guidewire once the guidewire has been positioned within the desired delivery site. The catheter 10 can thereafter be advanced along the guidewire in the manner of conventional “over-the-wire” balloon angioplasty catheters. A conventional guidewire having an exterior diameter of about 0.014 inches has a cross-sectional area of about 0.000154 inches, and a temporary stent 18 having an interior diameter of about 0.039 inches has an interior cross-sectional area of about 0.001194 inches. The cross-sectional area of the interior lumen 24 of stent 18, which can remain available for perfusion once a guidewire is in place, can therefore be about 0.00104 square inches.

The catheter 10 can be advanced through the vascular system, along the guidewire, until the drug delivery balloon 40 is disposed adjacent the desired delivery site. Thereafter, a suitable inflation fluid such as a radiopaque solution can be introduced by way of lumen 14 into the inflation balloon 30 to press the delivery balloon 32 against the vascular wall. Although described herein in its drug delivery capacity, the catheter can alternatively be used to perform dilatation, as has previously been described.

Once the drug delivery balloon 40 is positioned adjacent the vascular wall, medication can be infused by way of lumen 16 in tubular body 12 and expelled through effluent ports 40 directly against the vascular wall. Medication can be introduced under gravity feed alone, or by way of a positive pressure pump, as desired by the clinician in view of such factors as drug viscosity, toxicity and desired delivery time.

In this manner, drugs can be permitted to be absorbed directly into the affected site, with a minimal amount of drug escaping into generalized circulation. The rate of drug delivery can be somewhat limited by the rate of absorption by the vascular wall, and delivery rates on the order of about 30 ml per hour to about 20 ml per minute can be used. Certain medications can be optimally delivered at much lower rates, such as 1 ml per day or lower. However, these rates can be modified significantly, depending upon the drug, and the extent to which “overflow” fluid is permitted to escape into the circulatory system.

In the drug delivery application, delivery of a sufficient amount of drug can require an extended period of time. Perfusion past the delivery balloon by way of temporary stent 18 minimizes the adverse impact on circulation due to the indwelling drug delivery catheter. Following infusion of the predetermined volume of drug, and optionally following a further “rinse” with a sufficient volume of N-saline to expel substantially all of the drug from the residual volume of lumen 16 and space between drug delivery balloon 32 and inflation balloon 30, the inflation balloon 30 can be deflated and the catheter can be withdrawn.

During the foregoing procedures, the guidewire (not illustrated) can either be removed or can be left in place, as will be understood by one of skill in the art. In general, cardiologists prefer to leave the guidewire in place so that the catheter can be withdrawn and replaced, or other catheters can be inserted.

In modified method, the catheter 10 can be utilized as a temporary stent for an observation period following percutaneous transluminal coronary angioplasty, atherectomy, laser ablation or any of a variety of other interventional catheter techniques and procedures. In some arrangements, the drug delivery balloon 32 can be omitted entirely, and the tubular body 12 can optionally be provided with only a single fluid lumen extending therethrough to provide communication with the interior of inflation balloon 30.

Following removal of an interventional therapeutic catheter, such as an angioplasty, atherectomy or laser ablation catheter, the temporary stent catheter 10 can be inserted along the guidewire or through an introduction sheath and disposed with the inflation balloon 30 at the previously treated site. Inflation balloon 30 can be inflated to the desired diameter to resist reocclusion during a post-procedure period. Alternatively, the catheter 10 can be introduced by way of an introduction sheath having a lumen with a large enough diameter to accommodate catheter 10. Such observation periods can vary depending upon the circumstances of the patient and the cardiologist, but generally range from about 30 minutes to about 24 hours. During this time, perfusion across the inflation balloon 30 can be permitted by way of temporary stent 18.

As described herein, the relative cross-sectional area of the lumen 24, even with an indwelling guidewire, can permit a significant degree of perfusion to occur. In addition, the longitudinal axis of lumen 24 can be generally concentric with or parallel to the longitudinal axis of the artery or vein in which the indwelling temporary stent is disposed. In this manner, the interruption of direction of blood flow can be minimized, thereby reducing the likelihood of damaging blood cells and introducing undesired turbulence.

In other arrangements, portions of the inflation balloon 30 and/or the drug delivery balloon 32 of the above-described catheter arrangements can carry, for example, a therapeutic agent that does not readily dissolve in an aqueous solution, such as, for example, Paclitaxel. Although specific therapeutic agents are described herein, the methods, devices, and other details disclosed herein are intended to be used or adapted for use with other therapeutic agents such as, but not limited to, catechins. Paclitaxel is a lipophylic agent and does not readily dissolve in aqueous solution. In some embodiments, Paclitaxel can be dissolved in ethanol or any other organic solvent that does not form micelles. A portion of the balloon 30, 32 can be dipped or otherwise coated in the solution and subsequently dried. Those of skill in the art will recognize that in other embodiments the therapeutic agent can be carried by the balloon 30, 32 in other manners, such as, for example, embedding the material, otherwise depositing the material on the surface of the balloon, and/or dispersing the material within the balloon material.

The coated balloon catheter can be used to dilate stenotic arterial lesions using standard intervention procedures. The balloons 30, 32 can be inflated to dilate the artery at the site of the lesion. While inflated, a bolus of release agent can be injected into the outer porous balloon 32 to release Paclitaxel from the coated portions of the balloon 30, 32 and facilitate its transport into the aortic wall. Solvents such as ethanol can be used to release Paclitaxel and dissolve it in solution. Alternatively or in addition, contrast medium including commercially available Visipaque 320, Omnipaque, or Magnevist can be used to improve the solubility of Paclitaxel.

The release of the therapeutic agent can be stopped or greatly reduced by injected saline into the outer balloon 32 to inhibit the dissolution process. An advantage of the above-described arrangement is that the release of the therapeutic agent can be controlled by a second agent (release agent) that is injected through the catheter. The dose of therapeutic agent released may be dependent on the potency of the release agent and the duration of application. This may be an advantage over existing methods of drug delivery via drug-coated surfaces, in which the delivery rate is predetermined by the composition and properties of the coating. The above-described catheter and method provides for improved and individualized dosing of the drug during the procedure. Furthermore, injection of excessive amount of release agent will not overdose the patient. Surplus amount of release agent will be washed into the blood stream without impacting the release of the therapeutic agent.

As mentioned, the above-described method and apparatus of placing a therapeutic agent on the surface of a drug delivery system and subsequently control the release the therapeutic agent with a release agent can be extended to other combinations of therapeutic drugs and release agent. For example, Lipophilic therapeutic agents do not readily dissolve in aqueous solutions such as blood. Organic solvents can be used to release lipophilic drugs from the surface of the delivery system.

In a modified arrangement, the therapeutic agents can be placed in a degradable polymeric carrier that is coated onto the drug delivery device. For example poly amino-ester is a known biodegradable polymer for drug delivery. The poly amino ester can be formulated such that it degrades rapidly at acidic pH. The therapeutic agent can be added to the poly amino ester and the drug delivery device can be coated with the solution. At physiological pH (pH 7.2) the coating is fairly stable retaining the therapeutic agent during the insertion and placement of the drug delivery system. Once the coated surface of the drug delivery device is positioned at the target site, a release agent of low pH (pH 5.0-6.5) can be injected to accelerate the degradation of the polymer and release the therapeutic agent. Using a pH-sensitive biocompatible drug carrier is only one example of biodegradable carriers that can be used to retain the drug. Other biodegradable carriers can be used with degradation rates dependent on other the physical properties of the solution besides pH. For example, carriers can be considered with a degradation rate dependent on the temperature or ionic concentration of the solution. The release agent can be designed accordingly to change the physical or chemical properties of the solution to increase the rate of degradation and, as such, the release of the therapeutic agent.

In a further arrangement, the therapeutic agent can be chemically bonded to the surface using reversible chemical bonds. For example, tannins including catechin can be added to the coating to retain the therapeutic agent using weak hydrogen bonds. The release agent can include substances with a higher affinity to tannin. Large proteins such as collagen are known to have a high affinity to tannins. Collagen would compete with and replace the therapeutic agent in the hydrogen bonds effectively releasing it into solution. Those of skill of the art will recognize that there are many chemical reactions that could be used to initially bond the therapeutic agent to a surface and subsequently release the agent by a second reaction that is initiated by administering a release agent. The release rate can be controlled by the concentration of the release agent and the duration of application.

The drug delivery device and method that utilizes the release agent described above is not limited to the catheter arrangements described in U.S. Pat. No. 5,295,962 and FIGS. 2-12 or as described herein. Those of skill in the art will recognize the principles of utilization of the therapeutic agent and release agent described above can be extended and applied to other devices the delivery of drugs into diseased locations in the body such as blood vessels, organs, and tumors. In such modified arrangements, the drug delivery device need not include the dual balloon arrangement described above but can use a single balloon and/or another type of expandable or moveable member. In such arrangements, the therapeutic agent can be retained on the surface of the device in contact or in vicinity to the treatment site and the therapeutic agent can be released from the surface by the administration of a second agent through the delivery device. The surface of the device can comprise a balloon or other moveable element. However, it is also anticipated that the surface can be a fixed or semi-fixed member.

Any of the embodiments of the angioplasty balloon catheter described herein can comprise both drug delivery and stent components or features. However, the embodiments of the angioplasty balloon catheter described herein are not so limited. In some embodiments, the angioplasty balloon catheter can comprise only a drug delivery system which can be used alone or in combination with other therapeutic procedures or devices. In addition, the angioplasty balloon catheter described herein can readily be used for angioplasty dilatation as well.

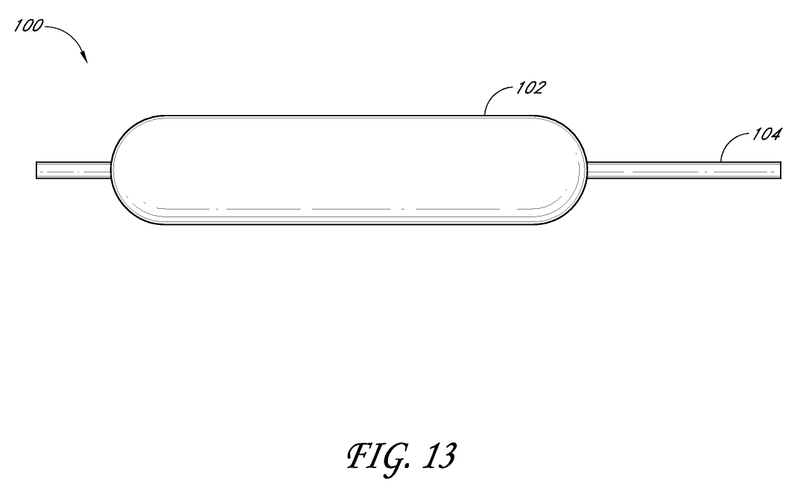

FIG. 13 is a schematic illustration of an embodiment of an angioplasty balloon catheter 100 that can comprise semi-elastic balloon 102 loaded with a therapeutic agent. With reference now to FIG. 13, the catheter 100 can comprise an inflatable balloon 100 that can be supported by a catheter shaft 104. The catheter shaft 104 can have a lumen formed therein to inflate the balloon 102. The general design of the balloon catheter 100 can be similar to that of existing or future developed balloon catheters used for angioplasty. These types of catheters are typically referred to in the literature as angioplasty catheters, PTCA catheters, and PTA catheters. However, as explained below, in some embodiments, the loading and release of a drug from the balloon 102 is a novel adaptation of the use of such catheters that provides significant advantages.

Drugs that can be considered to be delivered with the catheter 100 include, but are not limited to, anti-thrombogenic agents such as Heparin, magnesium sulfate, or anti-proliferation drugs such as Paclitaxel and Rapamycin, or photodynamic agents, or drugs to prevent extra-cellular matrix degeneration and/or promote cross-linking as described above, such as catechins and doxycycline. While Paclitaxel generally can have limited solubility in aqueous solutions, hydrophilic forms of Paclitaxel, for example, those that might be chelated to binding groups such as polyethylene glycol or polysaccharides, are considered to be appropriate for use with the embodiments described herein.

In some embodiments, the balloon can be made from a semi-elastic or elastic polymers or elastomer that is sensitive to a solvent, i.e. the balloon swells when exposed to a solvent. Balloon materials include but are not limited to latex, vinyl, silicone, polyurethane, and nylon. Solvents include but are not limited to acetone ethyl acetate, alcohol, and ethanol.

The desired therapeutic agent can be dissolved in the solvent in preparation for loading the balloon 102 with the agent. The concentration of agent can be chosen such that the agent has a therapeutic effect when loaded and subsequently released from the balloon 102. For example, in some arrangements, 2 mg/ml of Paclitaxel can be dissolved in 100% ethyl acetate. Additionally, in some arrangements, 5 mg/ml Catechin can be dissolved in 100% acetone. The concentration of the solvent in the solution can depend on the resistance of the balloon material to the solvent. For example, low-durometer polyurethane can have a low resistance to acetone, whereas nylon 6-6 can have a high resistance to acetone. Balloon material composed of multiple polymers, for example balloons that can be co-extruded or alternately dip cast such that a low durometer polymer is contained over a high durometer polymer, can also be used.

In some embodiments, to load the balloon 102 with the desired therapeutic agent, the balloon 102 can be immersed in the solution containing the solvent and the agent. The solvent preferably causes the balloon 102 to swell and thus facilitates the absorption of the agent into the balloon wall. The balloon 102 can be immersed either in a collapsed state, or in an inflated state at low pressure, or an inflated state at high pressure. Inflating the balloon 102 can expose the surface of the balloon more uniformly to the solvent. In some embodiments, it can be advantageous to inflate the balloon 102 to a high pressure to stretch the balloon material and increase the permeability of the balloon. In some embodiments, the balloon can be immersed in the solution for between one and five minutes to sufficiently load the balloon with the therapeutic agent. In some embodiments, the balloon can be immersed in the solution for a longer or shorter period of time. The balloon can be subsequently dried to flash off the solvent. The process of immersing the balloon in the solvent and drying can be repeated several times to increase the concentration of the agent in the balloon wall.

After the solvent is sufficiently removed from the balloon 102, the agent preferably remains substantially contained within the micro-structure of the balloon until the agent is released as described below. When the balloon 102 is inserted into a blood vessel, the agent will preferably not readily escape or be removed from the balloon material. In some embodiments, only small amounts of agent may be prematurely released in the blood stream.

After the balloon 102 has been inserted into the vasculature to the desired or target vessel or position in the vessel, the balloon 102 can then be inflated so as to abut against the vessel wall. The balloon 102 can be inserted into the vasculature using any suitable apparatus and/or technique either presently known in the field or later developed. When the balloon 102 is inflated in the target vessel against the vessel wall, the balloon material can be stretched and the permeability of its microstructure can be increased. At this point, when the balloon 102 is in an inflated configuration, the agent can be rapidly released from the balloon 102. At the same time, depending on the level of inflation of the balloon 102, the endothelial layer on the internal surface of the blood vessel can be stretched. It is well know that the endothelial cells do not generally stretch with the extra-cellular matrix. Stretching of the arterial wall generally creates gaps between the endothelial cells that act as channels for the agent to enter the extra-cellular matrix. Thus, balloon angioplasty can effectively temporarily increase the permeability of the endothelium for rapid drug delivery. In some embodiments, for the reasons mentioned above, it can therefore be advantageous to inflate the balloon 102 beyond the nominal diameter of the target vessel. This is in contrast to other proposed drug delivery balloon systems that are intended to merely make contact with or conform to the inner wall of the artery for drug delivery.

When the balloon 102 is inflated, the balloon material can be potentially exposed to high stresses. Angioplasty balloons are typically inflated to 2-12 atm. The balloon can therefore be made from material, in addition to other materials disclosed herein, with high tensile strength such as polyethylene (“PE”) or nylon. In PTCA, rigid balloons that do not stretch and, therefore, that retain there shape when inflated can be advantageous. Using balloons that do not stretch can allow the clinician or operator to pre-select the exact balloon diameter best suited for a particular blood vessel. However, for drug delivery, a semi-elastic and elastic balloon material can be preferred. Furthermore, the tensile strength of the balloon material can be compromised when exposed to a solvent. For example, polyurethane is known to crack after long exposure to a solvent. Thus, there exist competing design constraints for the construction of a drug-delivery balloon catheter as described here. Further, since different polymers have different molecular structures, the micro-porosities of these materials will vary.

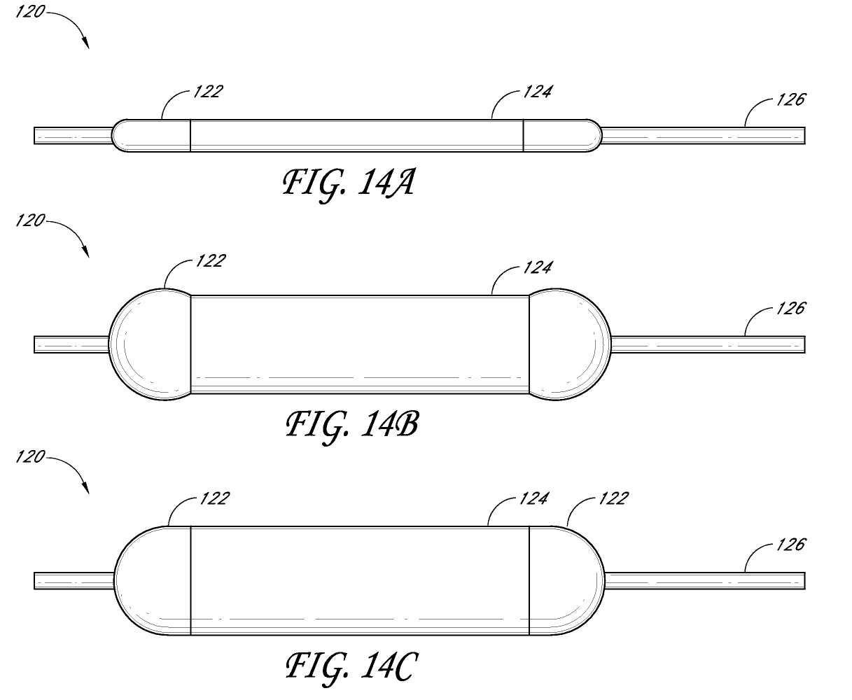

In some embodiments, the design constraints can be overcome by configuring the balloon catheter to comprise two co-axially positioned balloons. In particular, in some embodiments, as illustrated in FIGS. 14A-16B, the balloon catheter can comprise an inner balloon and an outer balloon, with the inner balloon being partially or substantially fully contained within the outer balloon.

FIG. 14A is a schematic illustration of an embodiment of an angioplasty balloon catheter 120 comprising a PTA balloon 122 covered by a tubular sleeve 124 that can be loaded with a therapeutic agent, showing the balloon 122 in a collapsed state. FIG. 14B is a schematic illustration of the embodiment of the angioplasty balloon catheter 120 shown in FIG. 14, showing the PTA balloon 122 in a partially inflated state. FIG. 14C is a schematic illustration of the embodiment of the angioplasty balloon catheter 120 shown in FIG. 14, showing the balloon 122 in a fully inflated state. In some embodiments, as is illustrated in FIGS. 14A-14C, the catheter 120 can be configured such that the inner PTA balloon 122 is only partially contained within the outer balloon 124, such that the outer balloon 124 covers only a portion of the inner balloon 122. However, in some embodiments, the catheter 120 can be configured such that the inner PTA balloon 122 is substantially completely contained within the outer balloon 124.

In some embodiments, the outer balloon 124 can be formed from a semi-elastic or elastic material, and can be configured to be loadable with a therapeutic agent, similar to the embodiments described above. The inner balloon 122 can be a standard angioplasty balloon, and can be inflatable during angioplasty. In some embodiments, the inner balloon 122 can formed or configured so as to possess a suitable level of mechanical strength to withstand the high inflation pressure. In this arrangement, the outer balloon 124 can be expanded by the inner balloon 122. The relaxed diameter of the outer balloon 124 can be less than that of the inner balloon 122, specifically when the inner balloon 122 is formed from a rigid or semi-rigid material. In some embodiments, in the above described configuration, the outer balloon 124 can be stretched for rapid drug release when the inner balloon 122 is inflated to its nominal size.

To deflate the outer balloon for catheter retraction, the shoulders of the inner balloon 122 and the outer balloon 124 can be bonded together as described by U.S. Pat. Nos. 5,295,962 and 5,569,184, the contents of both of which are hereby incorporated by reference as if fully set forth herein. Alternatively, the outer balloon 124 can be attached to a separate inflation lumen formed in the catheter body 126 to control the pressure of the outer balloon 124 independent of the pressure of the inner balloon 122. Other details regarding therapeutic agent delivery are set forth in U.S. Pat. Nos. 5,368,566, 5,569,215, and 5,542,926, the contents of all of which are hereby incorporated by reference as if fully set forth herein.

In some embodiments, the outer balloon 124 can be constructed from a highly elastic material such as, but not limited to, latex having stretch ratios over 100%. The outer balloon 124 can be configured so that, in a collapsed or unexpanded position, the cross-sectional size or profile of the outer balloon 124 can be approximately the same as or similar to the profile or cross-sectional size of the catheter body 126. Additionally, in some embodiments, when the inner balloon 122 is deflated, the outer balloon 124 can collapse or compress to its original diameter without the application of additional deflation to the inner or outer balloons 122, 124.

In some embodiments, the balloon catheters described above can be formed so as to define two or more separate layers of material (not illustrated). In some embodiments of this arrangement, the balloon catheter can be configured such that the inner layer of material can be more rigid than the outer layer of material, so that the inner layer provides mechanical strength to the balloon, and the outer layer is configured to be loadable with, and deliver, the therapeutic agent.

The method of loading and releasing a therapeutic agent from a layer of polymer or elastomer can be incorporated into a variety of drug delivery systems. For example, a stent graft can be constructed with a graft that contains a therapeutic agent. Upon deployment, the stent can expand the graft, thereby releasing the agent.

Another aspect of some of the embodiments disclosed herein relates to the delivery of the agent into the smooth muscle cells within the aortic wall. For example, Paclitaxel generally must enter the cell in order to down-regulate its proliferation. Paclitaxel generally does not easily pass through the cell membrane. In some of the embodiments disclosed herein, polyethylene imide (“PEI”) can be used with Paclitaxel. PEI has a high affinity to Paclitaxel and can act as a carrier of the Paclitaxel to carry the Paclitaxel across or through the cell membrane. Alternatively, in some embodiments, other chelating agents can be used, such as, but not limited to, ethylene diamine tetraacetic acid (“EDTA”).

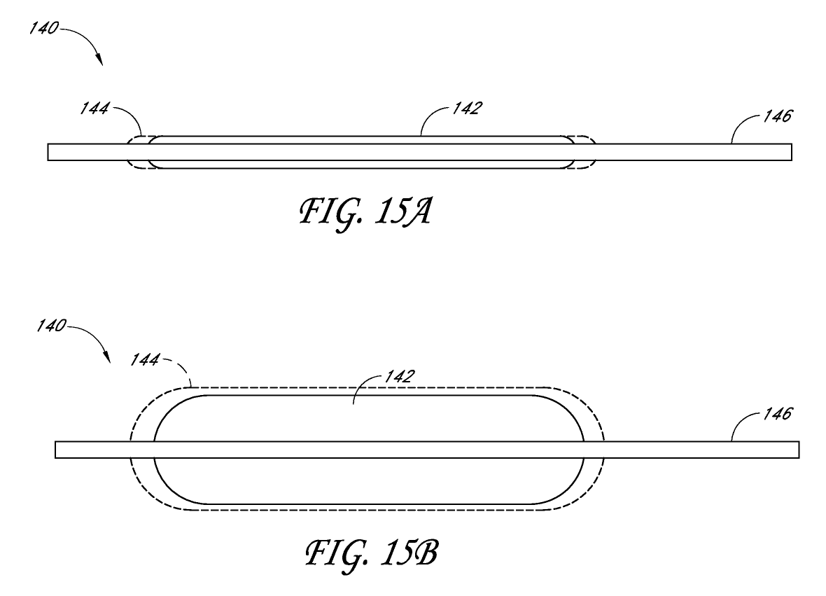

FIG. 15A is a schematic illustration of another embodiment of an angioplasty balloon catheter 140 comprising an inner PTA balloon 142 and an outer balloon 144 that can be loaded with a therapeutic agent. In this Figure, the balloons 142, 144 are shown in a collapsed state. FIG. 15B is a schematic illustration of the embodiment of the angioplasty balloon catheter 140 shown in FIG. 15A, showing the balloons 142, 144 in a fully inflated state. With reference to FIGS. 15A, 15B, the angioplasty balloon catheter 140 illustrated therein can be used to treat longer lesions of a diseased blood vessel, such as in the case of diffuse atherosclerotic disease. Long lesions may otherwise require the use of multiple drug-delivery balloons.

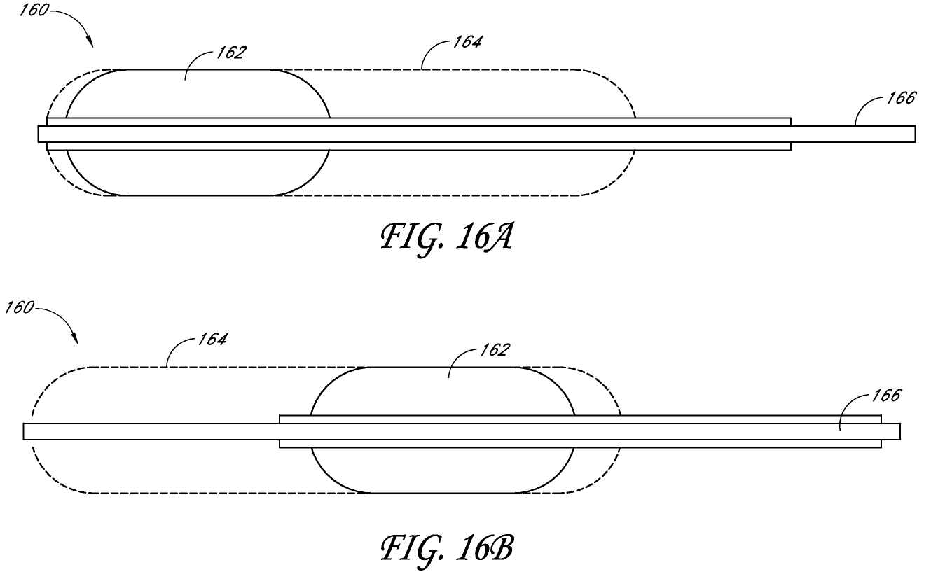

FIG. 16A is a schematic illustration of another embodiment of an angioplasty balloon catheter 160 with an inner PTA balloon 162 and an outer balloon 164 that can be loaded with a therapeutic agent. In FIG. 16A, the inner balloon 162 is shown inflating the distal section of the outer balloon 164. FIG. 16B is a schematic illustration of the embodiment of the angioplasty balloon catheter 160 shown in FIG. 16A, showing the inner balloon 162 inflating the proximal section of the outer balloon 164. FIGS. 16A and 16B illustrate an alternative embodiment of an angioplasty balloon catheter for treatment of long lesions. With reference to FIGS. 16A and 16B, in some embodiments, the outer balloon 144 can be significantly longer than the inner balloon 122. The outer balloon 164 can be sized and configured to span the full length of a lesion or other disease portion to be treated. The catheter 160 can be configured such that the inner balloon 162 can move axially inside the outer balloon 164, allowing for the inflation of discrete sections of the outer balloon 164. In this arrangement, a long lesion can be treated with one balloon catheter 160 by sequentially inflating each of the sections of the outer balloon 164.

Another aspect of this disclosure relates to the surface tension of the balloon material used for delivery of the therapeutic agent. The balloon can be configured to have a high surface tension to repel absorption of aqueous solutions. For that reason, an organic solvent can be used to enhance the transport of the therapeutic agent into the ePTFE or balloon matrix. When the balloon is inserted into the blood vessel, in some embodiments, it can be exposed to the blood stream. Because blood is an aqueous solution, it generally does not readily penetrate into the ePTFE or balloon matrix. Only the agent on the surface of the balloon can potentially be removed. The bulk of the agent can remain within the porous structure of the balloon material. Blood can, however, penetrate into the matrix and extract the agent when the surface tension is reduced. This can be done by injecting an organic solvent at the time of balloon inflation.

The surface tension can be reduced by applying physical pressure to the surface of the balloon. When the balloon is pressed against the vessel wall, pressure can be exerted onto the balloon surface, breaking the surface tension and allowing blood serum to penetrate into the matrix and extract the agent. Therefore, in some embodiments, the balloon can be expanded beyond the diameter of the blood vessel so as to allow blood serum to penetrate into the matrix and extract the agent.

Additionally, in some embodiments, any of the balloons described herein can be formed from a latex material. The following method can be used to load a latex balloon with a therapeutic agent. However, the method is not limited to latex and can be applied to other elastic materials such as silicone and polyurethane. In some embodiments, polyethylene glycol (PEG) can be added to the latex emulsion. Various molecular weights of PEG can be used. In some embodiments, a lower molecular weight PEG can be used to improve the dispersion of PEG in the latex emulsion. In some embodiments, PEG with a molecular weight between approximately 100 and approximately 1000, or between approximately 200 and 400 can be used. The concentration of PEG in the emulsion can be between approximately 0.05% and approximately 5%, or between approximately 0.5% and approximately 2%.

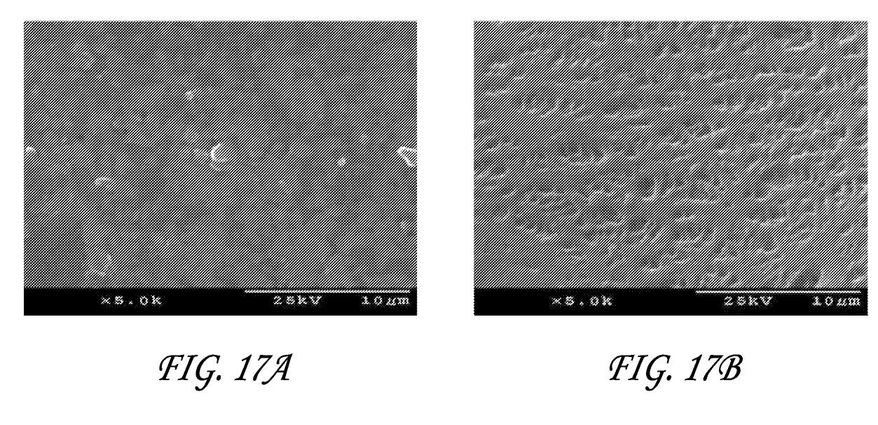

In some embodiments, the PEG can interfere with the cross-linking of the latex, thereby locally disrupting the micro structure of the latex. The PEG can subsequently be removed from the cured balloon with an organic solvent. FIG. 17A shows an SEM image at 5.0 k magnification of a latex surface prepared with 1% PEG having molecular weight of between approximately 380-420. The surface is generally smooth with the indication of some granulation. FIG. 17B shows an SEM image at 5.0 k magnification of the surface of the latex shown in FIG. 17A, stretched to about 400% of its original dimensions. Micropores can be created where PEG interfered with the cross-linking of the latex. When the stretched latex balloon is emerged in a solution containing an organic solvent and a therapeutic agent, the solution can penetrate into the micro pores. The solution can then be evaporated, leaving the agent in the pores. The latex can then be collapsed, trapping the agent in the microstructure. The balloon can be inserted into the blood stream in a collapsed state. Preferably only small amounts of agents will elute from the balloon in the collapsed state. Once the balloon is inflated and contacts the wall of the blood vessel, serum can enter the micro pores and transport the agent into the vessel wall.

In some embodiments, an advantage of the elastic pores in the balloon is that the agent can be physically trapped in the balloon. In some embodiments, no chemical bonding of the agent to the balloon, which can alter the properties of the agent, is required. Also, in this arrangement, no chemical bonding is needed to be overcome to release the agent from the balloon. It may be advantageous to deliver the therapeutic agent with other agents that increase the dissolution of the agent in serum, or the transport of the therapeutic agent into the wall, or increase the permeability of the extracellular matrix or cell membranes, or increase the residence time of the agent in the vessel wall. For example chelating agents such as PEI and EDTA can increase the dissolution of Paclitaxel, increase the affinity to cell and the extracellular matrix, and increase cell permeability. Any of these additional agents can be added to the solution containing the therapeutic agent and loaded into the microstructure of the balloon. Any suitable agents can be loaded into the balloon sequentially using separate solutions for each agent.

In other embodiments, Paclitaxel and a chelating agents such as PEI or EDTA can be loaded and delivered with the microporous balloon. The chelating agent can enhance the transport of Paclitaxel to and into the targeted smooth muscle cells.

It will be apparent to one of ordinary skill in the art of medical balloon manufacturing that various balloon materials and agents can be used to create a porous matrix. For example, in some embodiments, salt microparticles can be added to the emulsion and dissolved and removed after curing. Alternatively the cured material can be exposed to a strong organic solvent such as acetone to break down the molecular structure at the surface of the balloon. In some embodiments, micropores can be created that substantially enlarge when the balloon material is stretched from its collapsed state to its inflated state.

As mentioned, other delivery techniques other therapeutic agent are contemplated herein. For example, in some arrangements, the desired portion of the artery can be treated by briefly isolating the desired region from blood flow and releasing a desired quantity of the therapeutic agent in that region. In particular, in some embodiments, this can be achieved by expanding a pair of angioplasty or other suitable balloons on either side of the desired region of the vasculature. The first balloon can be positioned upstream of the desired region, and the second balloon can be positioned downstream the desired region. Using any suitable techniques, the region between each of the balloons can be filled with the therapeutic agent for a desired period of time. In some embodiments, the exposure time can be as short as one minute, or as long as 15 minutes or longer. With proper techniques and instruments, even longer exposure periods are attainable.

Additionally, in some embodiments, the cross-linking and other therapeutic agents disclosed above can be delivered by similar means as disclosed in U.S. Pat. No. 7,252,834 or by any other suitable means. Alternatively, in some embodiments, the desired therapeutic agents can be delivered into the body with a drug delivery catheter. Embodiments of suitable drug delivery catheters are described in U.S. Pat. Nos. 5,295,962 and 5,569,184. In some embodiments, the catechin or other therapeutic agent can be directly released from an implanted graft or stent with short-term release characteristics. For example, without limitation, the agent can be coated onto the graft material of the stent graft. A permeable polymer coating can be applied to the graft containing the agent. In the case of an ePTFE graft, the agent can be deposited directly into the porous graft as described in U.S. Provisional Patent Application No. 61/012,579, titled “Graft with Therapeutic Agent” which is hereby incorporated by reference as if fully set forth herein.

Vascular ePTFE grafts can be manufactured by extruding PTFE tubing, sintering the extruded material to obtain mechanical strength and mechanically stretching and expanding the material to obtain the desired final geometrical and mechanical specification. Improvements to the surface biocompatibility contemplated by prior art typically include the application of a surface coating to the final ePTFE graft.

In some embodiments, an ePTFE graft can be loaded with catechins, specifically EpiGalloCatechin Gallate (EGCG), to decrease its thrombogenicity. The molecular structure of some catechins are shown in FIG. 1. Other flavenoids and catechin compounds that are known to have a therapeutic effect can also be used. To introduce the agent into the ePTFE structure, the agent can be dissolved in acetone. Other organic solvent systems such as alcohol and acetate may also be considered. The solvents should be able to penetrate the ePTFE without damaging its structure. ePTFE is highly resistant to organic solvents and therefore well suited as a drug carrier. The graft can be submerged in the acetone solution containing the agent. Alternatively, in some embodiments, only the lumen of the ePTFE graft can be filled with acetone solution containing the desired agent or agents. A pressure gradient can be created across the graft by pressurizing the graft or applying vacuum to the outside of the graft to facilitate penetration of the acetone solution. For example, EGCG has a low molecular weight (less than 1000) and can be readily transported into the porous matrix of the ePTFE graft by the acetone. The graft can be then dried to flash off the actone. The EGCG remains in the matrix.

The concentration of EGCG in acetone can be in some embodiments between about 0.01% and about 10%, and in other embodiments between about 0.1% and about 1%. The desired concentration in a particular application can be dependent on the desired release rate and desired anti-thrombogenic surface properties. The graft can be submerged in the acetone solution for 30 seconds to several hours, preferably between 1 minute and 10 minutes. The acetone solution can be applied multiple times to increase the concentration of EGCG in the graft.

In some embodiments, the ePTFE graft can be a tubular endovascular graft that can be supported by a support structure, such as the endovascular grafts described in U.S. Pat. No. 6,733,523, entitled “Implantable Vascular Graft,” filed on Jun. 26, 2001, the entirety of which is hereby incorporated by reference as if fully set forth herein. Those of skill of the art will recognize that various embodiments and/or aspects of the grafts disclosed in U.S. Pat. No. 6,733,523 can be combined with the various features described herein to produce additional embodiments of an endovascular graft having certain features and advantages according the present disclosure.

Another aspect of the current disclosure is the anti-hyperplastic properties of EGCG. When surgical grafts are connected to blood vessels, the blood vessel can be exposed to increased stresses at the anastomosis. This is particularly true for veins in A-V shunt procedures. The mechanical stresses cause smooth muscle cell proliferation and migration into the vessel lumen. The migrating smooth muscle cells effectively reduce the size of the vessel lumen and can completely obstruct the lumen. This process is referred to as intimal hyperplasia. It is one of the primary failure modes of small diameter grafts. In the initial sequence of the process, Matrix Metalloproteinase (MMP) is released from the smooth muscle cells to break down the collagen matrix and path the way for cell migration. EGCG suppresses the activity of MMPs and hence reduce cell migration into the lumen. U.S. Pat. No. 6,214,868 sets forth additional details on the relevant mechanism of EGCG. In some embodiments of the disclosure, EGCG is released from the ePTFE graft into the adjacent tissue at the anastomosis site. Grafts can be sutured or stapled to the blood vessel. The pressure created by the sutures or staples forces body fluid into the porous structure of the ePTFE. EGCG dissolves readily in aqueous solutions such as blood and is rapidly transported into the tissue. EGCG also has a high affinity to protein, specifically collagen, preventing a wash-out into the blood stream.

Another aspect of the present disclosure relates to the high surface tension of ePTFE. The ePTFE material repels aqueous solutions. For that reason an organic solvent may be needed to transport the therapeutic agent into the ePTFE matrix. When the ePTFE graft is implanted, it can be exposed to blood and saline. Because these fluids are aqueous solutions, they cannot substantially penetrate into the ePTFE matrix. Therefore, only the agent on the surface of the ePTFE graft can be readily removed. The bulk of the agent can stay within the porous structure of the graft. Blood can only penetrate into the matrix and extract the agent when the surface tension is reduced. This can be done by adding a solvent. Alternatively, the surface tension can be reduced by applying physical pressure to the surface. As mentioned earlier, sutures and staples used to perform the anastomosis press the tissue against the graft and break the surface tension. The surface tension can also be reduced by blood elements contacting the surface of the ePTFE. Proteins are known to reduce surface tension. When platelets adhere to the surface of the ePTFE, they also enhance the release of catechin, which in return inhibit platelet aggregation.

In another embodiment of the disclosure, the porosity of the graft can be varied along the graft to optimize drug release. Along the inner layer of the graft a small pore size may be desirable to minimize platelet adhesion. At the anastomosis sites a large pore size may be advantageous to maximize the loading of EGCG for the prevention of hyperplasia. The concentration of EGCG in the graft can also be increased at the anastomosis sites by multiple applications of the acetone solution to the ends of the graft.

In some embodiments, the ePTFE graft can consist of several layers. In some embodiments, only the inner blood-contacting layer is treated with EGCG. The untreated outer layer can promote blood coagulation and adhesion to the blood vessel.

It is understood that many other therapeutic agents that can be dissolved in an organic solvent can be applied to the ePTFE graft. They can include, but are not limited to, Heparin, Paclitaxel, Rapamycin, and doxycycline.

Using any of the delivery methods or devices described herein, catechins can be delivered into the wall of the blood vessel. Preferably the catechin contains at least EGCG and ECG, preferably between approximately 20% and approximately 60% EGCG, and between approximately 5% and approximately 30% ECG. In some embodiments, the inflation time of the balloon can be between approximately 10 seconds and approximately 60 minutes or more, preferably between approximately 1 min and approximately 15 minutes. This can be different from long-term application of agents eluting from a device.

Although the inventions have been disclosed in the context of a preferred embodiments and examples, it will be understood by those skilled in the art that the present disclosure extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It can be also contemplated that various combinations or subcombinations of the specific features and aspects of the embodiments can be made and still fall within the scope of the invention. Accordingly, it should be understood that various features and aspects of the disclosed embodiments can be combine with or substituted for one another in order to form varying modes of the disclosed invention. Thus, it can be intended that the scope of the present disclosure herein disclosed should not be limited by the particular disclosed embodiments described above.

What is claimed is:

1. A method for stabilizing an extra cellular matrix layer in the vascular system of the body comprising:
placing a vascular catheter adjacent to the extra cellular matrix layer;
delivering a solution comprising a bioflavonoid to the extra cellular matrix layer with the vascular catheter; and
cross-linking protein in the extra cellular matrix layer.

2. The method of claim 1, wherein the protein is a collagen.

3. The method of claim 1, wherein the bioflavonoid is selected from the group consisting of: proanthocyanidin, catechin, epicatechin, epigallo catechin, epicatechin gallate, epigallocatechin gallate, quercetin, tannic acid, and any combination thereof.

4. The method of claim 1, wherein the bioflavonoid is epigallocatechin gallate.

5. The method of claim 1, wherein the bioflavonoid forms at least one hydrogen bond with the protein in the extra cellular matrix layer.

6. The method of claim 5, wherein the protein is a collagen.

7. The method of claim 6, wherein the extra cellular matrix layer is located in an aortic aneurysm.

8. The method of claim 1, wherein the solution has a pH less than 7.4.

9. The method of claim 1, wherein the solution contains a keotropic agent.

10. The method of claim 9, wherein the keotropic agent is Ca(OH)₂.

11. The method of claim 1, wherein the solution has a pH close to the isoelectric point of collagen or elastin.

12. The method of claim 1, wherein the extra cellular matrix layer is located in the aorta.

13. The method of claim 1, wherein the extra cellular matrix layer is located in an aortic aneurysm.

14. The method of claim 1, wherein the extra cellular matrix layer is the fibrous cap of vulnerable plaque.

15. The method of claim 1, wherein the solution is delivered to the extra cellular matrix layer using a stent graft.

16. The method of claim 15, wherein the stent graft comprises ePTFE.

17. The method of claim 1, wherein the solution is delivered to the extra cellular matrix layer using at least one expandable balloon configured to expand against the extra cellular matrix layer.

18. The method of claim 17, wherein at least one expandable balloon comprises latex.

19. The method of claim 17, comprising a first expandable balloon and a drug carrying member, the drug carrying member at least partially covering the expandable balloon and configured to carry the bioflavenoid and selectively release the bioflavenoid into the extra cellular matrix layer when the second member is positioned adjacent to the extra cellular matrix layer and expanded by the first expandable balloon.

20. A solution for treating an extra cellular matrix layer in situ, comprising a bioflavonoid and a keotropic agent.

21. The solution of claim 20, wherein the bioflavonoid is selected from the group consisting of: proanthocyanidin, catechin, epicatechin, epigallo catechin, epicatechin gallate, epigallocatechin gallate, quercetin, tannic acid, and any combination thereof.

22. The solution of claim 20, wherein the keotropic agent is Ca(OH)₂

23. The solution of claim 20, wherein the concentration of the bioflavonoid is between approximately 0.01% and approximately 5.0%.

24. The solution of claim 20, wherein the concentration of the keotropic agent is approximately 0.01% to approximately 1.0%.

25. The solution of claim 20, wherein the pH of the solution is less than approximately 7.4.

26. The solution of claim 20, wherein the extra cellular matrix layer is located in an injured or diseased artery.

27. The solution of claim 20, wherein the extra cellular matrix layer is located in a stenotic artery.

28. A method for reducing hyperplesia after injury of a blood vessel, comprising:
placing a vascular catheter adjacent to a wall of the blood vessel; and
delivering a solution containing a bioflavonoid to the wall of the blood vessel with the vascular catheter.

29. The method of claim 28, wherein the bioflavonoid is selected from the group consisting of: proanthocyanidin, catechin, epicatechin, epigallo catechin, epicatechin gallate, epigallocatechin gallate, quercetin, tannic acid, and any combination thereof.
